(19) United States
(12) Patent Application Publication
(10) Pub. No.: US 2020/0232993 A1
(43) Pub. Date: Jul. 23, 2020

(54) METHODS AND COMPOSITIONS FOR FLUORESCENT AND COLORIMETRIC PROTEIN QUANTITATION

(71) Applicant: PIERCE BIOTECHNOLOGY, INC., CARLSABD, CA (US)

(72) Inventors: Christopher ETIENNE, Oregon, WI (US); Ramesh GANAPATHY, Rockford, IL (US); Emily HALBRADER, Rockford, IL (US); Kelli FEATHER-HENIGAN, Rockford, IL (US); Aaron MCBRIDE, Rockford, IL (US); Brian WEBB, Roscoe, IL (US); Arjun PRASANNA, Rockford, IL (US)

(21) Appl. No.: 16/630,154

(22) PCT Filed: Jul. 16, 2018
(86) PCT No.: PCT/US2018/042299

§ 371 Date: Jan. 10, 2020

Related U.S. Application Data
(60) Provisional application No. 62/532,906, filed on Jul. 14, 2017.

(51) Int. Cl. G01N 33/68; G01N 33/52; G01N 21/64

(52) U.S. Cl. CPC G01N 33/6833; G01N 21/6428; G01N 33/52

(57) ABSTRACT

Compositions, kits and methods useful for determining the concentration of proteins or peptides in samples by fluorometric and/or colorimetric detection. Rapid quantitation of proteins and peptides or peptide mixtures by the present compositions, kits and methods provide one or more advantages such as but not limited to, methods that work at room temperature, no requirement for elevated temperatures or long incubation times, high sensitivity, low S/N background, detection in large and small sample volumes, detection in samples containing detergents and organic solvents.

### CROSS-REFERENCE TO RELATED APPLICATIONS

This application is a 371 U.S. National Phase Application of PCT/US18/42299 filed Jul. 16, 2018, which claims priority to and the benefit of U.S. Provisional Patent Application No. 62/532,906 filed Jul. 14, 2017.

### FIELD

Compositions, kits and methods useful for determining the concentration of proteins or peptides in samples by fluorometric and/or colorimetric detection.

### BACKGROUND

Protein quantitation is necessary prior to further isolation and characterization of a protein sample. It is a generally required step before submitting protein samples for chromatographic, electrophoretic, and immunochemical separation or analyses.

Protein quantitation is most commonly performed using colorimetric assays. Commercially available colorimetric protein and peptide solution quantitation methods include biuret (Gornall et al. J. Biol. Chem. 177 (1949) 751), Lowry (Lowry et al. J. Biol. Chem. 193 (1951) 265), bicinchoninic acid (BCA) (Smith et al. Anal. Biochem. 150 (1985) 76), Coomassie Blue G-250 dye-binding (Bradford, Anal. Biochem. 72 (1976) 248), and colloidal gold (Stoscheck, Anal. Biochem. 160 (1987) 301).

The biuret method is based on a protein forming a complex with cupric ions. Peptide nitrogen binds to copper (II) ion under alkaline conditions, producing a purple color. The absorption maximum of the product is 550 nm. The sensitivity is 1 mg protein/ml to 6 mg protein/ml. The biuret method is a relatively insensitive protein determination method compared to other commercial methods of colorimetric protein determination.

Another method combines the biuret reaction and the copper(1)-bathocuproine chelate reaction (Determination of Proteins by a Reverse Biuret Method Combined with the Copper-Bathocuproine Chelate Reaction. Clinica Chimica Acta., 216 (1993) 103-111). In this method, a sample protein forms a Cu²⁺-protein chelate complex (biuret reaction) during the first step. Excess Cu²⁺ is reduced to Cu⁺ by ascorbic acid, allowing Cu⁺ to form a Cu⁺-bathocuproine chelate complex during the second step. The amount of Cu⁺-bathocuproine chelate complex formed is inversely proportional to the protein concentration. This is a negative or indirect assay using a bathocuproine chelator to determine protein concentration. The Lowry method is a modified biuret reaction. It occurs in two steps: first, peptide bonds react with copper(II) ions under alkaline conditions, then Folin-Ciocalteau phosphomolybdic-phosphotungstic acid reduces to heteropolymolybdenum blue by copper-catalyzed oxidation of aromatic amino acids. The absorption maximum of the product is 750 nm. The Lowry method is more sensitive than the biuret method, with a linear sensitivity of 0.1 mg protein/ml to 1.5 mg protein/ml for bovine serum albumin (BSA). Certain amino acids, detergents, lipids, sugars, and nucleic acids interfere with the reaction. The reaction is pH dependent and pH should be maintained between pH 10 and pH 10.5.

The BCA method is related to the Lowry method in that peptide bonds in proteins first reduce cupric ion (Cu²⁺) to produce a tetradentate-cuprous ion (Cu¹⁺) complex in an alkaline medium. The cuprous ion complex then reacts with BCA (2 molecules BCA per Cu¹⁺) to form an intense purple color that can be measured at 562 nm. The BCA-Copper reaction is shown below:

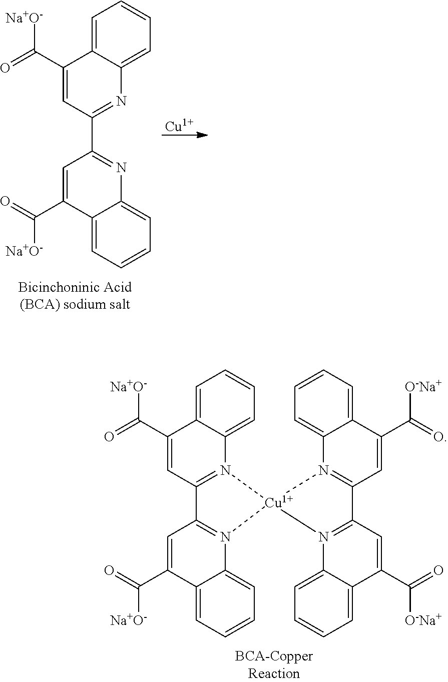

Because BCA is stable in alkaline medium, the BCA method can be carried out in one step, compared to two steps needed in the Lowry method. The BCA method better tolerates potential inhibitory or interfering compounds in the sample compared to the Lowry method. For example, up to 5% of each of sodium dodecyl sulfate (SDS), Triton X-100, and Tween-20 can be present and not interfere with the BCA method, compared to only 1% SDS, 0.03% Triton X-100, and 0.062% Tween-20 that can be present and not interfere with the Lowry method. The BCA method also has increased sensitivity and an expanded linear working range compared to the Lowry method.

A MICRO BCA™ Protein Assay Kit (Thermo Fisher Scientific) permits quantitation of dilute sample solutions (0.5 μg/ml to 20 μg/ml) by using larger sample volumes to obtain higher sensitivity. Despite the increased sensitivity, sample volume requirements limit or prevent its use for quantitation of many peptide samples.

A modified BCA assay to quantitate peptides (Kapoor et al. Analytical Biochemistry, 393 (2009) 138-140) acknowledges difficulties measuring peptide concentrations because of high interpeptide variation largely because of peptide hydrophobicity. The modified BCA method estimates peptide concentration by denaturing peptides by treatment at 95° C. for five minutes in the presence of SDS prior to incubation with the BCA working reagent. However, data below 500 μg/ml is very close to noise level and thus is not reliable.

U.S. Pat. No. 4,839,295 discloses using bicinchoninic acid as a chelator to detect proteins, measuring absorbance at 562 nm.

The colloidal gold method is the most sensitive among the colorimetric protein determination methods. Its sensitivity is about 2 μg/ml to 20 μg/ml protein. However, there is significant protein-to-protein variation. Protein binding to colloidal gold causes a shift in colloidal gold absorbance that is proportional to the amount of protein in solution. Most common reagents other than thiols and sodium dodecyl sulfate (SDS) are compatible with the colloidal gold method.

The Coomassie Blue G-250 dye-binding method is based on the immediate absorbance shift from 470 nm to 595 nm that occurs when Coomassie Blue G-250 binds protein in an acidic medium. Color development is rapid and the assay can be performed in ten minutes. The Coomassie Blue G-250 dye-binding method is comparatively free from interference by common reagents except detergents. There is moderate protein-to-protein variation and the method does not work well with peptides.

A total protein assay (Sozgen et al., Talanta, 68 (2006) 1601-1609 Spectrophotometric total protein assay with copper (II) neocuproine reagent in alkaline medium) uses copper(II)-neocuproine (Nc) reagent in alkaline medium with a hydroxide-carbonate-tartarate solution, with neocuproine as chelator. After 30 min incubation at 40° C., absorbance of the reduction product, Cu(I)—Nc complex, is read at 450 nm against a reagent blank. This assay has limited sensitivity because of the limited solubility of neocuproine in alkaline aqueous solution.

U.S. Pat. No. 5,693,291 discloses a quantitative protein method. The method is an indirect two-step method. It uses two reagents: reagent A (tartrate solution and copper sulfate) and reagent B (reducing agent, e.g., ascorbic acid, and bathocuprione disulfonate disodium salt as chelator). In the first step, copper ions complex with proteins present in a sample to form a complex wherein Cu²⁺ ions are reduced to Cu⁺. In the second step, excess Cu²⁺ ions, which are not reduced by complexing with sample proteins, are reduced by ascorbic acid to Cu⁺ ions. The Cu⁺ ions complex with bathocuproine to form a reddish brown color, which is detected colorimetrically. According to this method, larger quantities of proteins in a sample result in lower availability of Cu²⁺ ions that can be reduced by ascorbic acid in the second step, and therefore larger quantities of protein in a sample correspond to lower amount of color development by bathocuproine chelation. In other words, if there is no protein present in a sample, all the Cu²⁺ ions are reduced by ascorbic acid in the second step, thereby forming maximum color by bathocuproine chelation. Since the amount/quantity of protein correlates inversely with the amount/quantity/intensity of color formed, this assay is an indirect assay.

In addition, according to the indirect methods of U.S. Pat. No. 5,693,291, Reagent A contains 0.7 to 2 mmol/lCu²⁺ ions and 2 to 4 mmol/l tartrate in alkaline solution. Reagent B contains 1 to 1.5 mmol/l ascorbic acid and 0.5 to 0.8 mmol/l bathocuproine. The proportion of reagent A to reagent B is 1:8 to 1:12, i.e., 1 part reagent A to 8-12 parts reagent B. The combined volume of reagent A and reagent B is between 750 μl and 3000 μl, which is relatively large. Step one of the method mixes 100 μl Reagent A to 50 μl sample, followed by incubating at room temperature for 5 min to 60 min. Step two of the method adds 1 ml reagent B to the step one mixture, followed by brief mixing and reading at 485 nm. This negative or indirect assay quantitates protein by the difference in absorbance in the pre-versus post-bathocuproine chelated sample. It is thus less accurate than a positive or direct assay that quantitates protein directly. It also uses a large volume of standard protein to reagent (volume standard protein to reagent A is 1:1.6 to 1:2.4).

Compositions, kits and methods described in the present disclosure, overcome drawbacks in the art and provide additional benefits.

### SUMMARY

The present disclosure provides compositions, kits and methods for rapid quantitation of proteins and peptides or peptide mixtures that are amenable to fluorometric and/or colorimetric detection. Compositions, kits and methods provided herein for rapid quantitation of proteins and peptides or peptide mixtures provide one or more advantages such as, simple compositions, methods work rapidly, methods that work at room temperature, no requirement for elevated temperatures or long incubation times, high sensitivity, low S/N background, detection in large and small sample volumes, detection in samples containing detergents and organic solvents.

In some embodiments, the present disclosure provides a composition comprising: acetonitrile; and a reagent comprising or having the general formula (I):

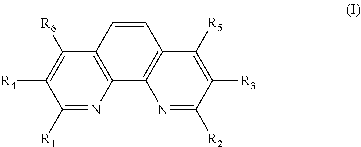

where, each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅); each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺); wherein when R₅ and R₆ is a phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and wherein the reagent is present in a hydrated, ((I) H₂O), or a non-hydrated form.

In some non-limiting examples, a reagent of general formula (I) has the molecular formula:

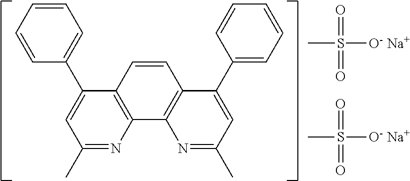

and is a hydrate or a non-hydrate form of the above structure.

In some non-limiting examples, a reagent of general formula (I) has the molecular formula:

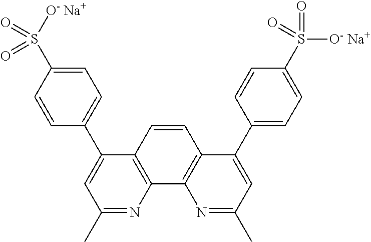

and is a hydrate or a non-hydrate form of the above structure.

In some non-limiting examples, a reagent of general formula (I) has the molecular formula:

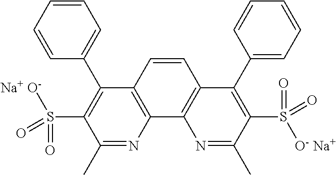

and is a hydrate or a non-hydrate form of the above structure.

In some non-limiting examples, a reagent of general formula (I) has the molecular formula:

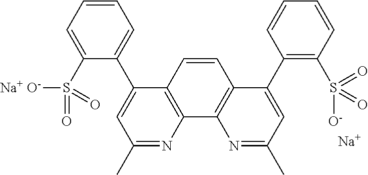

and is a hydrate or a non-hydrate form of the above structure.

In some embodiments, a composition of the present disclosure comprises a reagent having, comprising or of the general formula (I) and/or any one or more of the molecular formulae depicted above, including any combinations thereof.

In some embodiments, a composition of the present disclosure comprises acetonitrile, a reagent of the general formula (I) and/or any one or more of the molecular formulae depicted above including any combinations thereof.

In some embodiments, in a composition of the present disclosure, the concentration range of the reagent of formula (I) and/or any one or more of the molecular formulae depicted above, is from about 0.01 M to 0.1 M.

In some embodiments, in a composition of the present disclosure, the concentration range of acetonitrile is of from about 5%-30%. Acetonitrile concentration is measured as volume/volume % and can be 5%, 6%, 7%, 8%, 9%, 10%, 11%, 12%, 13%, 14%, 15%, 16%, 17%, 18%, 19%, 20%, 21%, 21%, 22%, 23%, 24%, 25%, 26%, 27%, 28%, 29%, 30% and includes values in-between.

In some embodiments, a composition of the present disclosure further comprises a tartrate. Tartarate can be sodium tartarate, potassium tartrate, or sodium potassium tartrate. In some embodiments, the concentration range of tartarate is from about 5.7 mM to about 22.7 mM and includes values in between.

In some embodiments, a composition of the present disclosure further comprises sodium bicarbonate or potassium bicarbonate.

In some embodiments, a composition of the present disclosure further comprises a buffer selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

In some embodiments, a composition of the disclosure comprises CAPS buffer. In some embodiments, a composition of the disclosure comprises CABS buffer. In some embodiments, a composition of the disclosure comprises borate buffer.

A composition of the disclosure, in some embodiments can further comprise copper. In some embodiments, copper can be added to a composition of the disclosure. The copper is preferably in a form that provides a source of Cu²⁺ ions. In some embodiments, copper is comprised in copper (II) sulphate, copper (II) bromide, copper (II) chloride, copper (II) fluoride, copper (II) perchlorate, copper (II) molybdate, copper (II) nitrate, copper (II) hydroxide, copper (II) tetrafluoroborate. In some embodiments, copper is at a concentration ranging from about 0.25 mM to about 0.5 mM.

Composition of the disclosure can have a pH ranging from about 11-12.2. In some embodiments a composition of the disclosure, has a pH of 11, 11.1, 11.2, 11.3, 11.4, 11.5, 11.6, 11.7, 11.8, 11.9, 12, 12.1, or 12.2.

In some embodiments, compositions of the disclosure comprise a stop solution for stopping reactions. Exemplary stop solutions include but are not limited to acetic acid, citric acid, ascorbic acid, formic acid, hydrochloric acid or sulphuric acid.

In some embodiments, compositions of the disclosure comprise a signal enhancer comprising a metal chelator added to enhance fluorescent emissions. Exemplary signal enhancers comprise one or more of Nitrilotriacetic acid (NTA) —N(CH₂CO₂H)₃, Ethylenediamine tetra acetic acid (EDTA), Iminodiacetic acid (IDA) or triscarboxymethyl ethylene diamine (TED).

In some embodiments, compositions of the disclosure comprise a signal enhancer added to enhance fluorescent emissions and a stop solution. Exemplary signal enhancers include but are not limited to, metal chelators, Nitrilotriacetic acid (NTA) —N(CH₂CO₂H)₃, Ethylenediamine tetraacetic acid (EDTA), Iminodiacetic acid (IDA) or triscarboxymethyl ethylene diamine (TED). Exemplary stop solutions include but are not limited to acetic acid, citric acid, ascorbic acid, formic acid, hydrochloric acid or sulphuric acid.

In some embodiments, the present disclosure provides a method for determining protein or peptide concentration in a sample comprising: (a) combining the sample with the components listed below to form a mixture, the components comprising: copper; acetonitrile; and a reagent having the general formula (I) depicted below:

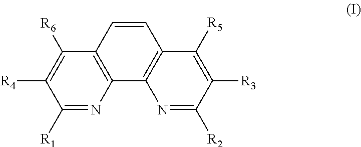

where, each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅); each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺); wherein when R₅ and R₆ is a phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and wherein the reagent is present in a hydrated, ((I) H₂O), or a non-hydrated form; (b) incubating the mixture under conditions sufficient to form a colored complex; and (c) measuring the fluorescence change by excitation of the colored complex at a first wavelength and measuring fluorescent emission at a second wavelength OR (c) by measuring absorbance of the colored complex.

In some embodiments, while measuring fluorescence, a first wavelength at which the colored complex is excited is between 450 nm to about 480 nm. In some embodiments, while measuring fluorescence, a second wavelength at which fluorescent emissions are measured (following excitation at a first wavelength), is between 660 nm to about 730 nm. In some embodiments, while measuring fluorescence, a second wavelength at which fluorescent emissions are measured (following excitation at a first wavelength), is between 510 nm to about 580 nm.

A fluorescence change or a fluorescent emission is typically measured or determined by a fluorometer.

In some embodiments, measuring the fluorescence of the colored complex is a direct indicator of protein or peptide concentration in the sample. A direct indicator of protein or peptide concentration in a sample corresponds to a method where the amount of fluorescence measured is directly proportional to the amount/quantity/concentration of protein or peptide in the sample. In some embodiments, when the colored complex formed in step (b) of the method described above, is excited at a first wavelength in the range of 450 nm-480 nm and when fluorescent emissions are measured at a second wavelength in the range of 660 nm-730 nm, the change in fluorescence is a direct measurement of protein or peptide concentration in the sample.

In some embodiments, measuring the fluorescence is an indirect indicator of protein or peptide concentration in the sample. An indirect indicator of protein or peptide concentration in a sample corresponds to a method where the amount of fluorescence measured is inversely proportional to the amount/quantity/concentration of protein or peptide in the sample. In some embodiments, when the colored complex formed in step (b) of the method described above, is excited at a first wavelength in the range of 450 nm-480 nm and when fluorescent emissions are measured at a second wavelength in the range of 510 nm to about 580 nm, the amount or quantity or concentration of protein or peptide in the sample, indirectly correlates with the fluorescence measured.

In embodiments of the method, when step (c) comprises measuring the absorbance of the colored complex, the absorbance or colorimetric change is typically measured or determined by a spectrophotometer or an automated microplate reader, such as but not limited to Genesys, Spectronic, Evolution, or NanoDrop™ spectrophotometer (all instruments by Thermo Fisher Scientific). In some embodiments, measuring the absorbance of the colored complex is done at 450 nm to 500 nm. Measuring the absorbance of the colored complex is a direct indicator of protein or peptide concentration in the sample. A direct indicator of protein or peptide concentration in a sample corresponds to a method where the amount of absorbance measured is directly proportional to the amount/quantity/concentration of protein or peptide in the sample.

In some embodiments, a method of the disclosure, further comprises determining protein or peptide concentration in the sample by comparing the fluorescence OR the absorbance measured in step (c) with the fluorescence OR the absorbance measured of at least one control sample containing a known concentration of a protein or peptide. Control samples having a pre-determined concentration range of a protein or peptide are known as protein standards or peptide standards. In some embodiments, a standard curve comprising the fluorescence emissions or absorbance of a protein standard or a peptide standard at various concentrations are determined by the present method and the intensity of fluorescent emissions or absorbance values at various concentrations of protein or peptide are plotted. The concentration of an unknown sample protein or peptide is then determined by the present method and the fluorescence emission or absorbance of the unknown sample is plotted on the standard curve to determine its concentration. Commonly used protein standards include but are not limited to Bovine Serum Albumin (BSA), purified antibodies such as Rabbit IgG, Mouse IgG etc. Commonly used peptide standards include but are not limited to tryptic digests of Bovine Serum Albumin (BSA), tryptic digests of Protein A, Protein A/G, HeLa Cell lysates etc. A protein standard or a peptide standard is a protein, a peptide, a peptide mixture, or a protein digest of any kind where the concentration has been pre-determined by a method known in the art.

In some embodiments, a method of the disclosure further comprises adding a stop solution after step (b) and prior to step (c). Exemplary stop solutions include but are not limited to one or more of acetic acid, citric acid, formic acid, hydrochloric acid, or sulphuric acid. Stop solutions can be added at a time decided by a person performing a method of the disclosure to provide uniformity of signal by stopping the reaction or preventing further formation of colored complex at a given time (such as 5 minutes, or anywhere between 0-5 minutes) for all the samples tested as well as for any protein/peptide standard tested. In some embodiments, a stop solution maintains the signal in a detectable range. In some embodiments, a stop solution is added while measuring protein concentration using a fluorometer.

In some embodiments, a method of the disclosure further comprises adding a signal enhancer after step (b) and prior to step (c). Enhancers can enhance the fluorescent signal to optimum detectable levels. Exemplary enhancers include but are not limited to metal chelators, Nitrilotriacetic acid (NTA), Ethylenediamine tetraacetic acid (EDTA), Iminodiacetic acid (IDA) or triscarboxymethyl ethylene diamine (TED).

In some embodiments, a method of the disclosure further comprises comprising adding a stop solution and an enhancer together after step (b) and prior to step (c).

In some embodiments of the methods of the disclosure, the sample is a biological sample or an artificially generated sample having one or more proteins or peptides whose concentration is to be determined. A biological sample can comprise a cell, a tissue, a lysate of a cell, a tissue, organs, a bodily fluid including but not limited to blood, plasma, serum, bone marrow, cerebrospinal fluid, spinal tap, saliva, nasal fluids, urine and feces.

Proteins, whose concentration is determined by a method of the disclosure can be a polypeptide, a gylcopeptide, a multimeric protein, a phosphoprotein, any post-translationally modified protein and combinations thereof. In some embodiments of the methods of the disclosure, the peptide, whose concentration is determined, is three amino acids or longer.

In some embodiments of a method of the disclosure, copper added to a sample provides a source of Cu²⁺ ions. The copper can be comprised in copper (II) sulphate, Copper (II) bromide, copper (II) chloride, copper (II) fluoride, Copper (II) perchlorate, copper (II) molybdate, copper (II) nitrate, copper (II) hydroxide, copper (II) tetrafluoroborate. In some embodiments of the method, the concentration range of copper added to the sample is from about 0.25 mM to about 0.5 mM.

In some embodiments of a method of the disclosure, the concentration of acetonitrile is 5%, 10%, 15%, 20%, 25%, 30% including values in between. The concentration of acetonitrile is measured in volume/volume %.

In some embodiments of a method of the disclosure, where the sample is further combined with tartrate. In some embodiments, the sample can be combined with sodium tartarate, potassium tartrate, or sodium potassium tartrate. In some embodiments, the concentration range of tartarate is from about 5.7 mM to about 22.7 mM, including values in between. In some embodiments of a method of the disclosure, the sample is further combined with sodium bicarbonate.

In some embodiments of a method of the disclosure, the sample is further combined with a buffer selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

In some embodiments of a method of the disclosure, the sample is further combined the CAPS buffer. In some embodiments of a method of the disclosure, the sample is further combined the CABS buffer. In some embodiments of a method of the disclosure, the sample is further combined the borate buffer.

In some embodiments of a method of the disclosure, the mixture has a pH range of from about 11-12.2. The pH of the mixture can be 11, 11.1, 11.2, 11.3, 11.4, 11.5, 11.6, 11.7, 11.8, 11.9, 12, 12.1, or 12.2.

In some embodiments of a method of the disclosure, the incubating is at room temperature. Room temperature is a temperature in the range of from about 18° C. to about 26° C., and encompasses temperatures including 18° C., 19° C., 20° C., 21° C., 22° C., 23° C., 24° C., 25° C. and 26° C. and includes temperatures in between these values. Room temperature can range of from about 20° C. to about 24° C. and in some embodiments room temperature can be about 22° C.

Some embodiments provide a rapid protein or peptide concentration detection method, where the colored complex forms and can be measured either colorimetrically or as a fluorescent emission in less than 25 minutes, less than 10 minutes, in 5 minutes, in less than 5 minutes, in 4 minutes, in 3 minutes, in 2 minutes in 1 minute, less than 1 minute, in 45 seconds, in 30 seconds, in 15 seconds, in less than 15 seconds and instantaneously.

Proteins or peptides can be detected in concentrations of from 20 μg/ml to 2000 μg/ml by methods of the disclosure.

In some embodiments, the sample volume that can be used to detect protein or peptide concentration by the present methods is about 5 μl to about 20 μl, about 5 μl, from about 10 μl to 20 μl, from about 15 μl to from 20 μl, and about 200 μl.

Sample comprising a plurality of proteins or peptides can be used for protein or peptide concentration determination by the present methods.

In some embodiments of the method, a reagent of general formula (I) has a molecular formula of one or more of the molecules depicted below, including:

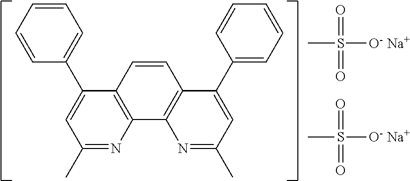

and a hydrate or a non-hydrate form of the above;

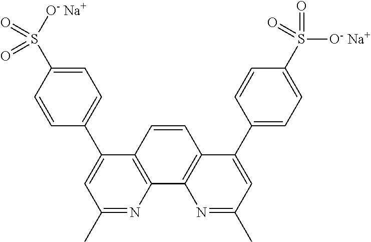

and a hydrate or a non-hydrate form of the above;

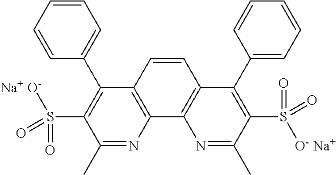

and a hydrate or a non-hydrate form of the above;

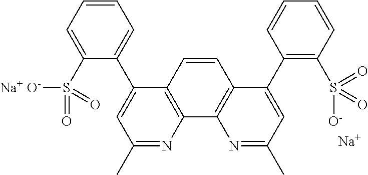

and a hydrate or a non-hydrate form of the above.

In some embodiments the method is amenable to analyze a sample that is in an aqueous solvent, an organic solvent, and combinations thereof. A sample that can be analyzed by methods of the disclosure can comprise at least one of an organic solvent, a detergent, and/or a reagent to improve protein or peptide solubility or stability. Exemplary detergents include but are not limited to, one or more of Triton X-100, Triton X-114, NP-40, Tween 80, Tween 20, CHAPS, and SDS. In some embodiments, the sample can comprise detergents such as but not limited to 5% Triton X-100, 5% Triton X-114, 5% NP-40, 5% Tween 80, 5% Tween 20, 5% CHAPS, 5% SDS.

A method of the disclosure can further comprise analyzing the proteins or peptide(s) whose concentration is determined further by one or more method including chromatography, electrophoresis, immunoassays, mass spectrometry, nuclear magnetic resonance (NMR), or Infrared (IR) spectroscopy.

The present disclosure also provides kits comprising: 1) a composition comprising acetonitrile and a reagent having the general formula (I) depicted below:

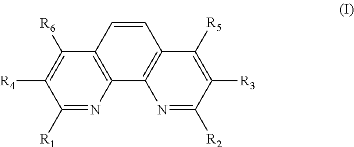

where, each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅); each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺); wherein when R₅ and R₆ is phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and wherein the reagent is present in a hydrated, ((I) H₂O), or a non-hydrated form; and 2) copper; each ingredient contained in one or more separate containers.

In some embodiments of a kit of the present disclosure, the concentration range of the reagent of formula (I) is from about 0.01 M to about 0.1 M; the concentration range of acetonitrile is from about 5%-30%; and the concentration range of copper is from about 0.25 mM to about 0.5 mM. Ranges include all values in between.

The composition comprised in a kit of the disclosure can further comprise one or more ingredients including a tartarate selected from sodium tartarate, potassium tartarate, sodium bicarbonate, potassium bicarbonate, sodium potassium bicarbonate, and one or more buffers selected from 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

In some embodiments of a kit of the disclosure, the concentration of tartrate is from about 5.7 mM to about 22.7 mM, the concentration of sodium bicarbonate, potassium bicarbonate or sodium potassium bicarbonate is from about 0.01-0.2 M. The pH of the components of a kit of the disclosure, in use, is from about 11-12.2.

In some embodiments, a kit of the disclosure further comprises one or more stop solution for stopping fluorescence signal (or colorimetric signal) from exceeding detectable signal levels, comprising acetic acid, citric acid, ascorbic acid, formic acid, hydrochloric acid or sulphuric acid. A stop solution will be packaged in a separate container in the kit.

In some embodiments a kit of the disclosure further comprises one or more an agent to enhance fluorescent emissions. Example signal enhancing agents include one or more metal chelators such as but not limited to of EDTA, IDA, NTA and TED. An enhancing agent will be packaged in a separate container in the kit, for use if signal enhancement is desired.

In some embodiments of a kit of the disclosure, one or more stop solution and one or more signal enhancer can be packaged together in a separate container.

While specific advantages have been disclosed hereinabove, it will be understood that various embodiments may include all, some, or none of the previously disclosed advantages. Other technical advantages may become readily apparent to those skilled in the art in light of the teachings of the present disclosure. These and other features of the present teachings will become more apparent from the detailed description in sections below.

### BRIEF DESCRIPTION OF THE DRAWINGS

One or more embodiments of the present disclosure may be better understood in reference to one or more the drawings below. The skilled artisan will understand that the drawings, described below, are for illustration purposes only. The drawings are not intended to limit the scope of the present teachings in any way.

FIG. 1 shows protein quantitation using a method and a composition according to certain embodiments provided herein, showing the detection of protein standard BSA at various concentrations at room temperatures at varying incubation times, according to one embodiment of the disclosure;

FIG. 2 shows protein quantitation using a method according to certain embodiments provided herein compared to the commercial BCA™ method, and depicts a great improvement in time over BCA™ and temperature of incubation, according to one embodiment of the disclosure;

FIG. 3 shows protein quantitation of several lysates using a method and a composition according to certain embodiments provided herein compared to the commercial BCA™ method, according to one embodiment of the disclosure;

FIG. 4 shows protein quantitation using an embodiment of the present methods provided herein, compared to the commercial BCA™ method and compared to Theoretical determinations of protein quantity, according to one embodiment of the disclosure;

FIG. 5 shows rapid protein quantitation using an embodiment of the present method using colorimetric detection and an embodiment of a composition provided herein, at various times including in as less as one minute, according to one embodiment of the disclosure;

FIG. 6 shows rapid protein quantitation using an embodiment of the present method provided herein, providing greater signal in lesser time as compared to the commercial BCA™ method, according to one embodiment of the disclosure;

FIG. 7 shows effect of varying acetonitrile concentrations on measuring protein concentrations using exemplary compositions provided herein in an exemplary method, according to one embodiment of the disclosure;

FIG. 8 shows effect of varying acetonitrile concentrations on measuring protein concentrations using certain exemplary compositions and methods provided herein, according to one embodiment of the disclosure;

FIG. 9 shows protein quantitation measured using an embodiment of the present compositions and methods provided herein, compared when measured by absorbance detection or measured by fluorescence detection, according to one embodiment of the disclosure;

FIG. 10 demonstrates and compares the stop effectiveness of several stop solutions on one embodiment of the methods provided herein to quantitate proteins using fluorometric measurements, according to one embodiment of the disclosure;

FIG. 11A & FIG. 11 B demonstrates and compares the stop effectiveness of several stop solutions on one embodiment of the methods provided herein to quantitate proteins using fluorometric measurements, according to one embodiment of the disclosure;

FIG. 12 demonstrates and compares the use of signal enhancers on one embodiment of the methods provided herein to quantitate proteins using fluorometric measurements, according to one embodiment of the disclosure;

FIG. 13 demonstrates and compares the stop effectiveness of several HCl stop solutions on one embodiment of the methods provided herein to quantitate proteins using fluorometric measurements, according to one embodiment of the disclosure;

FIG. 14A and FIG. 14B demonstrates and compares the stop effectiveness of several acid stop solution concentrations and volumes on one embodiment of the methods provided herein to quantitate proteins using fluorometric measurements, according to one embodiment of the disclosure;

FIG. 15 compares the stop effectiveness of several solutions on one embodiment of the methods provided herein to quantitate proteins using fluorometric measurements, according to one embodiment of the disclosure;

FIG. 16 compares the stop effectiveness of several solutions on one embodiment of the methods provided herein to quantitate proteins using fluorometric measurements, according to one embodiment of the disclosure;

FIG. 17 depicts a time line of start of fluorescence signal detection by a fluorometer for one embodiment of a method of the disclosure; and

FIG. 18 shows protein quantitation using a method and several exemplary compositions according to certain embodiments provided herein, showing the detection of protein standard BSA at various concentrations compared with the commercial BCA method, according to one embodiment of the disclosure.

### DETAILED DESCRIPTION

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range therebetween unless otherwise specifically stated. For example, “from 0.2 to 0.5” means 0.2, 0.3, 0.4, 0.5; ranges therebetween such as 0.2-0.3, 0.3-0.4, 0.2-0.4; increments there between such as 0.25, 0.35, 0.225, 0.335, 0.49; increment ranges there between such as 0.26-0.39; and the like.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

While several protein and peptide quantitation methods are known in the art, the present disclosure provides compositions, kits and methods for rapid quantitation of proteins and peptides, wherein the methods can be performed, according to some embodiments, in ten minutes or less, in five minutes or less, in 1 minute or less, at room temperature, and are amenable to fluorometric and/or colorimetric detection. Compositions, kits and methods provided herein for rapid quantitation of proteins and peptides or peptide mixtures provide one or more advantages such as, no requirement for elevated temperatures or long incubation times, high sensitivity, low S/N background, low variability in detection, detection in large and small sample volumes, ability to detect complex lysates and detection in samples containing detergents and organic solvents.

As discussed in sections above, U.S. Pat. No. 5,693,291 discloses an indirect method for protein quantitation. The method is an indirect two-step method, wherein, a sample is first reacted with a first reagent, Reagent A, comprising tartrate and copper sulfate. In the first step, copper ions of the copper sulfate, complex with proteins present in the sample, to form protein-copper complexes in which Cu²⁺ ions are reduced to Cu⁺. The protein-copper complex here is a protein-Cu⁺ complex. In the second step, excess Cu²⁺ ions, i.e., Cu²⁺ ions that are not reduced to Cu⁺ by forming protein-copper complexes, are then treated with Reagent B comprising ascorbic acid as a reducing agent and bathocuproine which is a Cu⁺ ion chelator. These excess (unbound) Cu²⁺ ions are reduced by ascorbic acid to form Cu⁺ ions. The Cu⁺ ions so formed are chelated by bathocuproine to form a bathocuprione-Cu⁺ complex that has a reddish brown color, which is detected colorimetrically. According to this method, larger quantities of proteins in a sample result in lower availability of free Cu²⁺ ions that can be reduced by ascorbic acid in the second step. Therefore larger quantities of protein in a sample correspond to lower amount of color development by bathocuproine chelation of Cu⁺ ions. Since the amount/quantity of protein correlates inversely with the amount/quantity/intensity of color formed, this assay is an indirect assay.

The disclosure of PCT Patent Application No: PCT/US2015/034960, published Dec. 17, 2015, having priority date Jun. 11, 2014, (U.S. application Ser. No. 14/734,678), by one or more of the present inventors, discloses a direct method for peptide and/or protein quantitation. According to PCT/US2015/034960, combining a sample comprising protein or peptides with copper sulfate, results in the formation of protein-copper/peptide-copper complexes where Cu²⁺ ions are reduced to Cu⁺. The protein-copper complex here is a protein-Cu⁺ complex. Bathocuprione then reacts with the Cu⁺ ions on the protein to form a Bathocuprione-Cu⁺-protein chelate that is orange brown in color. The absorbance is then measured at 450 nm to 500 nm. According to this method, since protein-Cu⁺ complex chelates with bathocuprione to form color that is measured, larger quantities or concentration of protein or peptide in the sample result in greater reduction of Cu²⁺ to Cu⁺ and greater amount of the Bathocuprione-Cu⁺-protein chelate being formed, which is measured spectrophometrically at 450 nm to 500 nm.

Rapisarada et al., in “Quenching of bathocuproine disulfonate fluorescence by Cu(I) as a basis for copper quantification,” in Analytical Biochemistry 307 (2002) 105-109, disclose methods for determining copper concentration in proteins using fluorescent properties of uses bathocuprione disulfonate disodium salt hydrate. This article describes linear quenching of bathocuproine disulfonate (BCS) emissions at 770 nm (λₑₓ 580 nm) by increasing concentrations of Cu(I), at neutral pH of 7.5, are described. The procedure for determining the total copper content in soluble proteins is described to have three steps: release of the copper at pH<1, neutralization in the presence of citrate which stabilizes the copper, and reduction of the copper to Cu(I) by ascorbate in the presence of the chelator BCS. Standard copper samples are run in parallel with the test samples, in the same medium and conditions. The amount of copper in the test samples is calculated from their emission at 770 nm (λₑₓ 580 nm) in comparison with the standard curve of BCS fluorescence versus copper concentration.

The present inventors have surprisingly found novel fluorescent properties of bathocuprione compounds that have been adapted in the presently disclosed compositions, kits and methods to quantitate proteins or peptides using fluorometric detection. Using compositions of the present disclosure, which are have fewer components, for example, which do not comprise reducing agents such as ascorbic acid, the present inventors have designed novel methods for quantitating proteins or peptides, using fewer and simplified steps, for example but not limited to, steps not requiring drastic changes in pH, not requiring elevated temperatures, in contrast to prior methods requiring incubation time of 5 minutes or less, nor requiring contacting samples with additional reducing agents such as ascorbic acid for reduction of Cu²⁺ to Cu⁺. In some embodiments, compositions, kits and methods of the present disclosure allow detection of protein or peptide concentration in alkaline conditions.

Surprisingly, the present inventors have also found that the present rapid methods using present compositions, can be detected either by spectroscopic detection methods or by fluorometric methods with equal accuracy and sensitivity. Accordingly, using the presently disclosed novel compositions, kits and methods, rapid reduction of Cu²⁺ to Cu⁺ is achieved by complexing with sample proteins or peptides, to form protein-Cu⁺ complexes or peptide-Cu⁺ complexes. Further, the new compositions allow for chelation of Cu⁺ ions in the protein-Cu⁺ complex by bathocuprione molecules in the same step (without the need for: changes in pH; and/or elevated temperature and/or extended incubation time), to form a protein-Cu⁺-bathocuprione chelate complex. This protein-Cu⁺-bathocuprione chelate complex can be excited at a first wavelength and fluorescent emissions measured at a second wavelength to determine the protein or peptide concentration using a fluorometer. The colored complex comprising protein-Cu⁺-bathocuprione chelate complex can also be measured colorimetrically using a spectrophotometer. This allows for a single assay format to be used across diverse detection platforms.

One or more advantages of a fluorescent protein or peptide quantitation method include, but are not limited to: speed of the assay and additionally being able to determine protein concentration either colorimetrically or using fluorescence measurements, or both. This has advantages such as providing an internal check of the results as one can calculate protein concentration colorimetrically and confirm the results using fluorescence. Alternatively, one could calculate protein or peptide concentration using the fluorescent mode and then confirm results using the colorimetric mode. Additional advantages provided by the present methods and compositions are that since hand held fluorescent devices such as but not limited to the Qubit™ platform can be used with these methods, measurement of protein or peptide concentrations in a field setting is possible. This is especially useful for applications which require measurements in a field setting (i.e., without access to a laboratory/clinic) such as Human Identification (HID), crime scene detection, clinical detection of proteins for diagnosis in rural settings, third world areas, battlefield situations, diagnosis of animal diseases in farms or ranches or in the wild, food safety testing, etc.

Compositions, kits and methods of the present disclosure allow rapid detection of protein or peptide concentration, in some embodiments, in 10 minutes or less, and in some embodiments in 5 minutes, in less than 5 minutes, in 4 minutes, in 3 minutes, in 2 minutes, in 1 minute, in less than one minute, in 45 seconds, in 30 seconds, in 15 seconds and instantaneously, at Room Temperature, fluorometrically or colorimetrically.

### Compositions:

Provided herein are compositions comprising: acetonitrile; and a reagent having or comprising general formula (I):

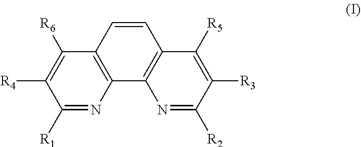

where, each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅); each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺); wherein when R₅ and R₆ is phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and wherein the reagent is present in a hydrated, ((I) H₂O), or a non-hydrated form.

In some embodiments, the molecule of formula (I) is 1,10-phenanthroline. In some non-limiting examples, a reagent of general formula (I) has one or more of the molecular formulae depicted below, including:

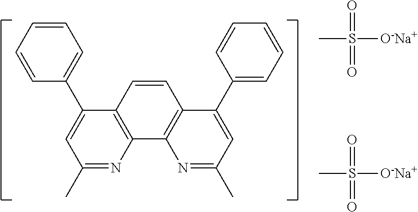

and a hydrate or a non-hydrate form of the above structure;

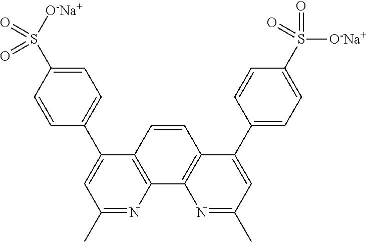

and a hydrate or a non-hydrate form of the above structure;

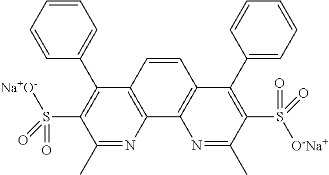

and a hydrate or a non-hydrate form of the above structure; and/or

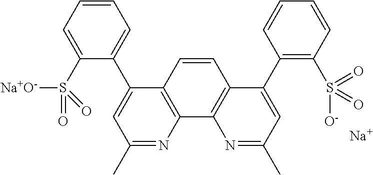

and is a hydrate or a non-hydrate form of the above structure.

Embodiments of compositions of the present disclosure comprises acetonitrile, and a reagent of the general formula (I) including for example, one or more of the molecular formulae depicted above, including any combinations thereof.

In some exemplary embodiments, compositions of the present disclosure comprise a reagent of formula (I) including one or more of the molecular formulae depicted above, in the range of from about 0.01 M to 0.1 M, including values in between, and acetonitrile in the concentration range of from about 5%-30%. Acetonitrile concentration is measured as volume/volume % and can be 5%, 6%, 7%, 8%, 9%, 10%, 11%, 12%, 13%, 14%, 15%, 16%, 17%, 18%, 19%, 20%, 21%, 21%, 22%, 23%, 24%, 25%, 26%, 27%, 28%, 29%, 30% and includes values in-between.

In some exemplary embodiments, a composition of the present disclosure further comprises tartrate. Tartarate can be sodium tartarate, potassium tartrate, or sodium potassium tartrate. In some embodiments, the concentration range of tartarate is from about 5.7 mM to about 22.7 mM, including values in between. In some embodiments, a composition of the present disclosure further comprises sodium bicarbonate or potassium bicarbonate.

A composition of the present disclosure further comprises a buffer selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

Structure of some of these buffers are set forth below. The chemical structure of some of these buffers is provided below. The structure of CAPS:

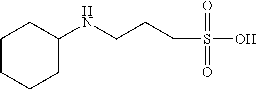 [SMILES: O=S(=O)(O)CCCNC1CCCCC1]

The structure of CABS:

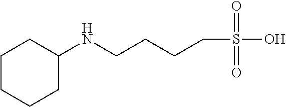 [SMILES: O=S(=O)(O)CCCCNC1CCCCC1]

The structure of CAPSO:

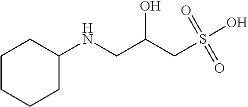 [SMILES: O=S(=O)(O)CC(O)CNC1CCCCC1]

The structure of TABS:

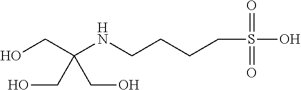 [SMILES: O=S(=O)(O)CCCCNC(CO)(CO)CO]

The structure of MOBS:

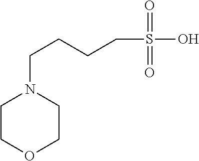 [SMILES: O=S(=O)(O)CCCCN1CCOCC1]

The structure of CHES:

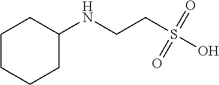 [SMILES: O=S(=O)(O)CCNC1CCCCC1]

The structure of AMPSO:

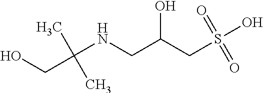

The structure of POPSO:

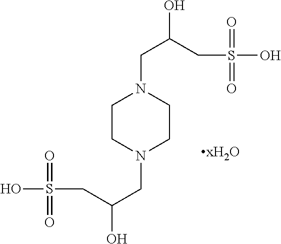 [SMILES: O=S(=O)(O)CC(O)CN1CCN(CC(O)CS(=O)(=O)O)CC1]

In some exemplary embodiments, a composition of the disclosure comprises a CAPS buffer or a CABS buffer or a borate buffer. In some non-limiting embodiments, a buffer as described above provides stability to the composition. In some non-limiting embodiments, a buffer as described above prevents the acetonitrile from precipitating from the solution and provides stability to the composition.

Composition of the disclosure can have a pH ranging from about 11-12.2. In some embodiments a composition of the disclosure, has a pH of 11, 11.1, 11.2, 11.3, 11.4, 11.5, 11.6, 11.7, 11.8, 11.9, 12, 12.1, or 12.2.

A composition of the disclosure, in some embodiments can further comprise copper. In some embodiments, copper can be added to a composition of the disclosure. The copper is preferably in a form that provides a source of Cu²⁺ ions. In some embodiments, copper is comprised in copper (II) sulphate, copper (II) bromide, copper (II) chloride, copper (II) fluoride, copper (II) perchlorate, copper (II) molybdate, copper (II) nitrate, copper (II) hydroxide, copper (II) tetrafluoroborate. In some embodiments, copper is at a concentration ranging from about 0.25 mM to about 0.5 mM and includes values in between.

In some embodiments, a composition of the disclosure comprises a stop solution for stopping reactions. Exemplary stop solutions include but are not limited to acetic acid, citric acid, ascorbic acid, formic acid, hydrochloric acid or sulphuric acid.

In some embodiments, a composition of the disclosure comprises a signal enhancer comprising a metal chelator added to enhance fluorescent emissions. Exemplary signal enhancers comprise one or more of Nitrilotriacetic acid (NTA) —N(CH₂CO₂H)₃, Ethylenediamine tetraacetic acid (EDTA), Iminodiacetic acid (IDA) or triscarboxymethyl ethylene diamine (TED).

In some embodiments, a composition of the disclosure comprises a signal enhancer added to enhance fluorescent emissions and a stop solution. Exemplary signal enhancers include but are not limited to, metal chelators, Nitrilotriacetic acid (NTA) —N(CH₂CO₂H)₃, Ethylenediamine tetraacetic acid (EDTA), Iminodiacetic acid (IDA) or triscarboxymethyl ethylene diamine (TED). Exemplary stop solutions include but are not limited to acetic acid, citric acid, ascorbic acid, formic acid, hydrochloric acid or sulphuric acid.

### Methods:

The present disclosure provides, in some embodiments, a method for determining protein or peptide concentration in a sample comprising the steps of: (a) combining the sample with the components listed below to form a mixture, the components comprising: copper; acetonitrile; and a reagent having or comprising general formula (I) depicted below:

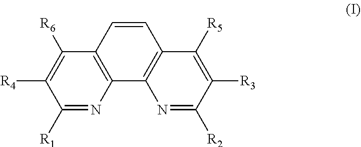

where, each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅); each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂) salt of sodium (Na⁺), carboxylate (—CO₂) salt of potassium (K⁺), and carboxylate (—CO₂) salt of lithium (Li⁺); wherein when R₅ and R₆ is phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂) salt of sodium (Na⁺), carboxylate (—CO₂) salt of potassium (K⁺) and carboxylate (—CO₂) salt of lithium (Li⁺); and wherein the reagent is present in a hydrated, ((I) H₂O), or a non-hydrated form; (b) incubating the mixture under conditions sufficient to form a colored complex; and (c) measuring the fluorescence change by excitation of the colored complex at a first wavelength and measuring fluorescent emission at a second wavelength OR (c) by measuring absorbance of the colored complex.

In some embodiments of the method, a reagent of general formula (I) has a molecular formula of one or more of the molecules depicted below, including:

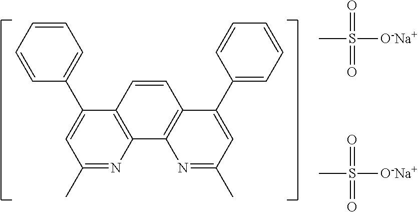

and a hydrate or a non-hydrate form of the above;

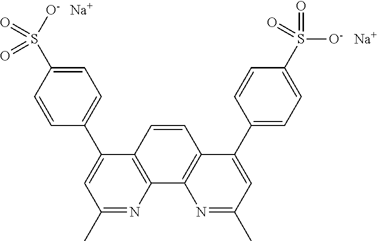 [SMILES: Cc1cc(-c2ccc(S(=O)(=O)[O-])cc2)c2ccc3c(-c4ccc(S(=O)(=O)[O-])cc4)cc(C)nc3c2n1 ; [Na+] ; [Na+]]

and a hydrate or a non-hydrate form of the above;

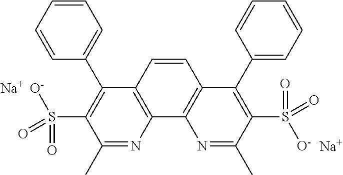 [SMILES: [Na+] ; Cc1nc2c(ccc3c(-c4ccccc4)c(S(=O)(=O)[O-])c(C)nc32)c(-c2ccccc2)c1S(=O)(=O)[O-] ; [Na+]]

and a hydrate or a non-hydrate form of the above;

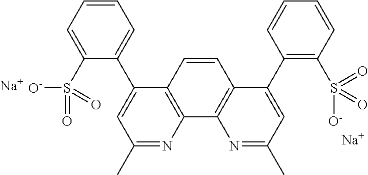 [SMILES: [Na+] ; Cc1cc(-c2ccccc2S(=O)(=O)[O-])c2ccc3c(-c4ccccc4S(=O)(=O)[O-])cc(C)nc3c2n1 ; [Na+]]

and a hydrate or a non-hydrate form of the above.

In some embodiments of a method of the disclosure, copper added to a sample provides a source of Cu²⁺ ions. The copper can be comprised in copper (II) sulphate, Copper (II) bromide, copper (II) chloride, copper (II) fluoride, Copper (II) perchlorate, copper (II) molybdate, copper (II) nitrate, copper (II) hydroxide, copper (II) tetrafluoroborate. In some embodiments of the method, the concentration range of copper added to the sample is from about 0.25 mM to about 0.5 mM, including values in between.

In some embodiments of a method of the disclosure, the concentration of acetonitrile is 5%, 10%, 15%, 20%, 25%, 30%, including values in between.

In some embodiments of a method of the disclosure, the sample is further combined with tartrate such as sodium tartarate, potassium tartarate or sodium potassium tartarate. In some embodiments of a method of the disclosure, the sample is further combined with sodium bicarbonate. In some embodiments of a method of the disclosure, the sample is further combined with a buffer selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO). In some embodiments of a method of the disclosure, the sample is further combined the CAPS buffer. In some embodiments of a method of the disclosure, the sample is further combined the CABS buffer. In some embodiments of a method of the disclosure, the sample is further combined the borate buffer.

In some embodiments of a method of the disclosure, the mixture has a pH range of from about 11-12.2. The pH of the mixture can be 11, 11.1, 11.2, 11.3, 11.4, 11.5, 11.6, 11.7, 11.8, 11.9, 12, 12.1, or 12.2.

In some embodiments of a method of the disclosure, the incubating is at room temperature. Room temperature is a temperature in the range of from about 18° C. to about 26° C., and encompasses temperatures including 18° C., 19° C., 20° C., 21° C., 22° C., 23° C., 24° C., 25° C. and 26° C. and includes temperatures in between these values. Room temperature can range of from about 20° C. to about 24° C. and in some embodiments room temperature can be about 22° C.

Some embodiments provide a rapid protein or peptide concentration detection method, where the colored complex forms and can be measured either colorimetrically or as a fluorescent emission in less than 25 minutes, less than 10 minutes, in 5 minutes, in less than 5 minutes, in 4 minutes, in 3 minutes, in 2 minutes in 1 minutes, in less than 1 minute, in 45 seconds, in 30 seconds, in 15 seconds or instantaneously.

While measuring fluorescence, a first wavelength at which the colored complex is excited is between 450 nm to about 480 nm. In some embodiments, while measuring fluorescence, a second wavelength at which fluorescent emissions are measured (following excitation at a first wavelength), is between 660 nm to about 730 nm. In some embodiments, while measuring fluorescence, a second wavelength at which fluorescent emissions are measured (following excitation at a first wavelength), is between 510 nm to about 580 nm.

A fluorescence change or a fluorescent emission is typically measured or determined by a fluorometer. Exemplary fluorometers that may be used are but not limited to Qubit™ (Thermo Fisher Scientific), Varioskan (Thermo Fisher Scientific), Quantus Fluorometer (Promega); Gemini (Molecular Devices), or by NanoDrop™ fluorometer (Thermo Fisher Scientific).

In some embodiments, measuring the fluorescence of the colored complex is a direct indicator of protein or peptide concentration in the sample. A direct indicator of protein or peptide concentration in a sample corresponds to a method where the amount of fluorescence measured is directly proportional to the amount/quantity/concentration of protein or peptide in the sample. In some embodiments, when the colored complex formed in step (b) of the method described above, is excited at a first wavelength in the range of 450 nm-480 nm and when fluorescent emissions are measured at a second wavelength in the range of 660 nm-730 nm, the change in fluorescence is a direct measurement of protein or peptide concentration in the sample.

The bathocuproine-Cu(I) complex, when excited at 450-480 nm, produces a fluorescent emission signal at 660-730 nm. The emission signal at 660-730 nm can only come from the bathocuproine-Cu(I) complex. The Cu(I) present is the result of protein/peptide reducing Cu(II) to Cu(I). Therefore, the amount of fluorescent emission signal at 660-730 nm is directly proportional to the amount of Cu(I) produced which is directly proportional to the amount of protein/peptide present.

In some embodiments, a method of the disclosure further comprises addition of one or more stop solutions to the colored complex, following a 0 minute to a 5 minutes incubation time, including incubation times in between this time range, and prior to measuring fluorescence. In some embodiments, the incubation time may be less than 1 minute, less than 5 minutes, 5 minutes, more than 5 minutes and may be 10 minutes or more. Exemplary stop solutions, according to the disclosure include acetic acid, hydrochloric acid, sulphuric acid. A stop solution stops the assay reaction and prevents further formation of the colored complex thereby keeping the emitted fluorescence in a detectable range. The stop solutions stop the assay by making the assay solution acidic. The stop solutions of the disclosure do not quench fluorescence.

In some embodiments, a method of the disclosure, further comprises adding an enhancing agent to enhance or improve the fluorescent emission signal. A chelator such as EDTA, NTA, IDA and TED can be added as enhancing agent.

In some embodiments, a method of the disclosure further comprises adding a stop solution after step (b) and prior to step (c). Exemplary stop solutions include but are not limited to one or more of acetic acid, citric acid, formic acid, hydrochloric acid, or sulphuric acid. Stop solutions can be added at a time decided by a person performing a method of the disclosure to provide uniformity of signal by stopping the reaction or preventing further formation of colored complex at a given time (such as 5 minutes, or anywhere between 0-5 minutes) for all the samples tested as well as for any protein/peptide standard tested. In some embodiments, a stop solution maintains the signal in a detectable range. In some embodiments, a stop solution is added while measuring protein concentration using a fluorometer.

In some embodiments, a method of the disclosure further comprises adding a signal enhancer after step (b) and prior to step (c). Enhancers can enhance the fluorescent signal to optimum detectable levels.

In some embodiments, a method of the disclosure further comprises comprising adding a stop solution and an enhancer together after step (b) and prior to step (c).

In some embodiments, measuring the fluorescence is an indirect indicator of protein or peptide concentration in the sample. An indirect indicator of protein or peptide concentration in a sample corresponds to a method where the amount of fluorescence measured is inversely proportional to the amount/quantity/concentration of protein or peptide in the sample. In some embodiments, when the colored complex formed in step (b) of the method described above, is excited at a first wavelength in the range of 450 nm-480 nm and when fluorescent emissions are measured at a second wavelength in the range of 510 nm to about 580 nm, the amount or quantity or concentration of protein or peptide in the sample, indirectly correlates with the fluorescence measured. While not being limited to theory, bathocuproine, when excited at 450 nm-480 nm, has a fluorescent emission at 510-580 nm. This fluorescent emission is from the bathocuproine that has not bound Cu(I). As the amount of free bathocuproine decreases, due to complexing with Cu(I) which is bound to proteins in a sample, fluorescent emissions at 510-580 nm decrease as well.

A method of the disclosure can further comprise determining protein or peptide concentration in the sample by comparing the fluorescence measured in step (c) with the fluorescence measured of at least one control sample containing a known concentration of a protein or a peptide. Control samples having a pre-determined concentration range of a protein or peptide are known as protein standards or peptide standards. In an exemplary embodiment, a standard curve comprising the fluorescence emissions of a protein standard or a peptide standard at various concentrations is determined by the present method and the intensity of fluorescent emissions at various concentrations of standard protein or standard peptide are plotted. The concentration of an unknown sample protein or peptide is then determined by the present method and the fluorescence emission of the unknown sample is plotted on the standard curve to determine its concentration. A protein standard or a peptide standard is a protein, a peptide, a peptide mixture, or a protein digest of any kind where the concentration has been pre-determined by a method known in the art.

Commonly used protein standards include but are not limited to Bovine Serum Albumin (BSA), purified antibodies such as Rabbit IgG, Mouse IgG, Goat, IgG, Sheep IgG or Human IgG etc. Commonly used peptide standards include but are not limited to tryptic digests of Bovine Serum Albumin (BSA), tryptic digests of Protein A, Protein A/G, HeLa Cell lysates etc. A protein standard or a peptide standard is a protein, a peptide, a peptide mixture, or a protein digest of any kind where the concentration has been pre-determined by a method known in the art.

In embodiments of the method, when step (c) comprises measuring the absorbance of the colored complex, the absorbance or colorimetric change is typically measured or determined by a spectrophotometer or an automated microplate reader. In some embodiments, measuring the absorbance of the colored complex is done at 450 nm to 500 nm. Measuring the absorbance of the colored complex is a direct indicator of protein or peptide concentration in the sample. A direct indicator of protein or peptide concentration in a sample corresponds to a method where the amount of absorbance measured is directly proportional to the amount/quantity/concentration of protein or peptide in the sample.

In some embodiments, a method of the disclosure further comprises determining protein or peptide concentration in the sample by comparing the absorbance measured in step (c) with the absorbance measured of at least one control sample containing a known concentration of a protein or peptide. As noted above, control samples having a pre-determined concentration range of a protein or peptide are known as protein standards or peptide standards. In some embodiments, a standard curve comprising the absorbance of a protein standard or a peptide standard at various concentrations are determined by the present method and the absorbance values at various concentrations of the standard protein or the standard peptide are plotted. The concentration of an unknown sample protein or peptide is then determined by the present method and the absorbance of the unknown sample is plotted on the standard curve to determine its concentration. A protein standard or a peptide standard is a protein, a peptide, a peptide mixture, or a protein digest of any kind where the concentration has been pre-determined by a method known in the art.

Various types of samples can be analyzed by methods of the disclosure to determine the protein or peptide concentration therein. For example, a sample can be a biological sample or an artificially generated/created sample having one or more proteins or peptides whose concentration is to be determined. Exemplary biological samples can include but are not limited to a cell, a tissue, a lysate of a cell, a tissue, organs, a bodily fluid including but not limited to blood, plasma, serum, bone marrow, cerebrospinal fluid, spinal tap, saliva, nasal fluids, urine and feces. Exemplary artificially generated/created samples can include but are not limited to synthetic protein or peptides generated in a lab.

The presently disclosed methods are amenable to analyze a sample that is in an aqueous solvent, in an organic solvent, and in solvents that are combinations of aqueous and organic solvents. For example, a sample containing a protein or peptide whose concentration is to be determined can be a lysate or a complex lysate in which components of the lysis buffer or solutions to dissolve or maintain the integrity of one or more protein or peptide components comprises an organic solvent, an aqueous solvent or both.

In other examples, a sample that can be analyzed by methods of the present disclosure can comprise at least one of an organic solvent, a detergent, and/or a reagent to improve protein or peptide solubility or stability. Exemplary detergents that may be comprised in a sample include but are not limited to, one or more of Triton X-100, Triton X-114, NP-40, Tween 80, Tween 20, CHAPS, and SDS. In some embodiments, the sample can comprise detergents such as but not limited to 5% Triton X-100, 5% Triton X-114, 5% NP-40, 5% Tween 80, 5% Tween 20, 5% CHAPS, 5% SDS.

Proteins, whose concentration is determined by a method of the disclosure can be a polypeptide, a gylcopeptide, a multimeric protein, a phosphoprotein, any post-translationally modified protein and combinations thereof. In some embodiments of the methods of the disclosure, the peptide, whose concentration is determined, is three amino acids or longer. A sample may therefore contain one or more of the above types of peptides or proteins.

Proteins or peptides can be detected in concentrations of from 20 μg/ml to 2000 μg/ml by methods of the disclosure.

In some embodiments, the sample volume that can be used to detect protein or peptide concentration by the present methods is about 5 μl to about 20 μl, about 5 μl, from about 10 μl to 20 μl, from about 15 μl to from 20 μl, and about 200 μl.

Sample comprising a plurality of proteins or peptides can be used for protein or peptide concentration determination by the present methods.

A method of the disclosure can further comprise analyzing the proteins or peptide(s) whose concentration is determined further by one or more method including chromatography, electrophoresis, immunoassays, mass spectrometry, nuclear magnetic resonance (NMR), or IR.

### Kits:

The present disclosure also describes kits for implementing the methods discussed herein and/or kits that contain compositions described herein. In one embodiment, the present disclosure also provides kits comprising: 1) a composition comprising acetonitrile and a reagent having or comprising general formula (I) depicted below:

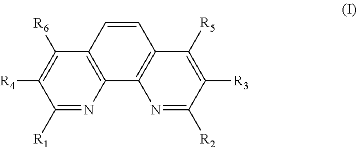

where, each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅); each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺); wherein when R₅ and R₆ is phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and wherein the reagent is present in a hydrated, ((I) H₂O), or a non-hydrated form; and 2) copper; each ingredient contained in one or more separate containers.

In some embodiments of a kit of the present disclosure, the concentration range of the reagent of formula (I) is from about 0.01 M to about 0.1 M; the concentration range of acetonitrile is from about 5%-50%; and the concentration range of copper is from about 0.25 mM to about 0.5 mM.

The composition comprised in a kit of the disclosure can further comprise one or more ingredients including a tartarate selected from sodium tartarate, potassium tartarate, sodium bicarbonate, potassium bicarbonate, sodium potassium bicarbonate, and one or more buffers selected from 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

In some embodiments of a kit of the disclosure, the concentration of tartrate is from about 5.7 mM to about 22.7 mM, the concentration of sodium bicarbonate, potassium bicarbonate or sodium potassium bicarbonate is from about 0.01M to 0.2 M. The pH of the components of a kit of the disclosure, in use, is from about 11-12.2.

In some embodiments, a kit of the disclosure further comprises one or more stop solution for stopping fluorescence signal (or colorimetric signal) from exceeding detectable signal levels, comprising acetic acid, citric acid, ascorbic acid, formic acid, hydrochloric acid or sulphuric acid. A stop solution will be packaged in a separate container in the kit.

In some embodiments a kit of the disclosure further comprises one or more an agent to enhance fluorescent emissions. Example signal enhancing agents include one or more metal chelators such as but not limited to of EDTA, IDA, NTA and TED. An enhancing agent will be packaged in a separate container in the kit, for use if signal enhancement is desired.

In some embodiments of a kit of the disclosure, one or more stop solution and one or more signal enhancer can be packaged together in a separate container.

Reagents and components of kits may be comprised in one or more suitable containers. A container may generally comprise at least one vial, test tube, flask, bottle, syringe or other container means, into which a component may be placed, and preferably, suitably aliquoted. Where there are more than one component in a kit they may be packaged together if suitable or the kit will generally contain a second, third or other additional container into which the additional components may be separately placed. However, in some embodiments, certain combinations of components may be packaged together comprised in one container means. A kit can also include a means for containing one or more compositions as set forth herein, and any other reagent containers in close confinement for commercial sale. Such containers may include injection or blow-molded plastic containers into which the desired vials are retained.

Some components of a kit are provided in one and/or more liquid solutions. Liquid solution may be non-aqueous solution, an aqueous solution, and may be a sterile solution.

Components of the kit may also be provided as dried powder(s). When reagents and/or components are provided as a dry powder, the powder can be reconstituted by the addition of a suitable solvent. It is envisioned that a suitable solvent may also be provided in another container means. Kits may also comprise a container means for containing a sterile, pharmaceutically acceptable buffer and/or other diluent.

A kit of the disclosure may also include instructions for employing the kit components and may also have instructions for the use of any other reagent not included in the kit. Instructions can include variations that can be implemented.

### EXAMPLES

Aspects of the present teachings can be further understood in light of the following examples, which should not be construed as limiting the scope of the present teachings in any way.

### Example 1

### Compositions & Methods for Rapid Assays Using Colorimetric Detection

Exemplary compositions of the disclosure were prepared as described below and tested according to an example methods of the disclosure comprising the steps of (a) combining a sample with acetonitrile, a reagent having the general formula (I), and copper to form a mixture; (b) incubating the mixture under conditions sufficient to form a colored complex; and (c) measuring absorbance of the colored complex at 450 nm to 500 nm as a direct indicator of protein or peptide concentration in the sample. The exemplary composition of the disclosure was tested by incubating the mixture (in step (b) of the method), at Room Temperature and for varying time intervals of from 5 minutes to 25 minutes.

A standard protein concentration calibration curve was generated using BSA standards at various concentrations ranging from 2 mg/mL to 0.125 mg/m L. The BSA standards were incubated in an exemplary composition of the disclosure, as described below, at different times in intervals of 5, 10, 15, 20 and 25 minutes at room temperature.

An exemplary composition of the disclosure, referred to as the working reagent herein, was made by adding 50 parts of Reagent A to 1 part of Reagent B. Components of Reagent A and Reagent B are listed in Table 1 below. The reagent B composition is in the working solution is 1.6 mg/mL.

TABLE 1
Exemplary Components for Reagent A and Reagent B according
to one Example of the Presently Disclosed Compositions

| Reagent A | Reagent B |
|---|---|
| 0.2M CAPS buffer | 80 mg/mL Cupric Sulfate |
| 0.2M Sodium Bicarbonate | |
| 0.8 mg/mL Sodium Tartrate | |
| 0.01M Bathocuproine disulfonic acid | |
| 10% Acetonitrile | |
| pH 11.8 | |

The assays were performed on a microplate. The standards were added in triplicates. Conditions used for the method are described in Table 2.

TABLE 2
Conditions and Parameters Used

| | |
|---|---|
| Volume of Protein Standards/Sample | 20 μL |
| Working Reagent v\Volume | 200 μL |
| Incubation temperature | Room Temp |
| Incubation time | 5 minutes, 10 minutes, |
| | 15 minutes, 20 minutes |
| | and 25 minutes |
| Absorbance at which microplate was read | 480 nm |

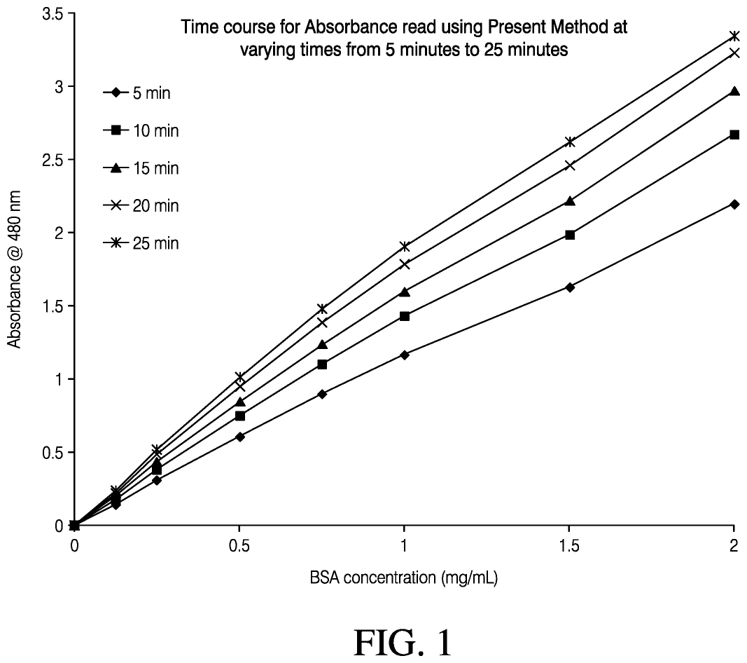

FIG. 1 shows results of the above exemplary method, which depict a BSA calibration curve obtained for the exemplary compositions of the disclosure as described above at different time points starting from 5 minutes to 25 minutes at Room temperature. The data shows a linear curve with increasing slope as the incubation time increases. Absorbance was detected using the present methods and present compositions as early as 5 minutes.

In the experiments, here and in the disclosure, while automated absorbance readings, using automated plate readers, are desirable for speed and convenience, the absorbance measurement of a reaction mixture measured in a cuvette can be alternatively used.

### Example 2

### Comparison of Bicinchoninic Add Assay (BCA) Versus the Present Methods and Compositions

BSA standards at various concentrations starting from 2 mg/mL down to 0.025 mg/mL were used to generate a calibration curve to compare the presently disclosed method using presently disclosed compositions and kits with the previous Thermo Scientific Pierce BCA™ Protein Assay Kit (referred to herein as the “BCA™ commercial method” or “BCA™”).

As noted earlier, the BCA™ Protein Assay combines the well-known reduction of Cu²⁺ to Cu¹⁺ by protein in an alkaline medium with the highly sensitive and selective colorimetric detection of the cuprous cation (Cu¹⁺) by bicinchoninic acid. The first step is the chelation of copper with protein in an alkaline environment to form a light blue complex. In this reaction, known as the biuret reaction, peptides containing three or more amino acid residues form a colored chelate complex with cupric ions in an alkaline environment containing sodium potassium tartrate. In the second step of the color development reaction, bicinchoninic acid (BCA) reacts with the reduced (cuprous) cation that was formed in step one. The intense purple-colored reaction product results from the chelation of two molecules of BCA with one cuprous ion. The BCA/copper complex is water-soluble and exhibits a strong linear absorbance at 562 nm with increasing protein concentrations. The BCA reagent is approximately 100 times more sensitive (lower limit of detection) than the pale blue color of the first reaction. The reaction that leads to BCA color formation is strongly influenced by four amino acid residues (cysteine or cystine, tyrosine, and tryptophan) in the amino acid sequence of the protein. However, unlike the Coomassie dye-binding methods, the universal peptide backbone also contributes to color formation, helping to minimize variability caused by protein compositional differences.

The working reagent for both the traditional BCA™ and the presently disclosed methods was made by adding 50 parts of Reagent A to 1 part of Reagent B from the Tables 1 and 3 for each method. See Table 1 for exemplary compositions for Reagent A and Reagent B of the present method. See Table 3 for components of Reagent A and Reagent B of the traditional BCA™ method. The methods were performed on a microplate. The standards were added in triplicates. The conditions described in Table 4 were used for each assay.

TABLE 3
Components for Reagent A and Reagent B
for the Traditional BCA ™ Method

| Reagent A | Reagent B |
|---|---|
| 0.161 M Sodium carbonate | 40 mg/mL Cupric Sulfate |
| 0.107 M Sodium Bicarbonate | |
| 1.6 mg/mL Sodium Tartrate | |
| 10 mg/mL Bicinchoninic acid | |
| pH 11.2 | |

TABLE 4
Conditions and Parameters used for each assay

| | BCA ™ | | Present Methods | |
|---|---|---|---|---|
| Standards/Sample volume | 25 | μL | 20 | μL |
| Working reagent volume | 200 | μL | 200 | μL |
| Incubation temperature | 37° C. | | Room Temp (range of from | |
| | | | about 18° C. to about 26° C.) | |
| Incubation time | 30 | minutes | 5 | minutes |
| Absorbance at which | 562 | nm | 480 | nm |
| plate was read | | | | |

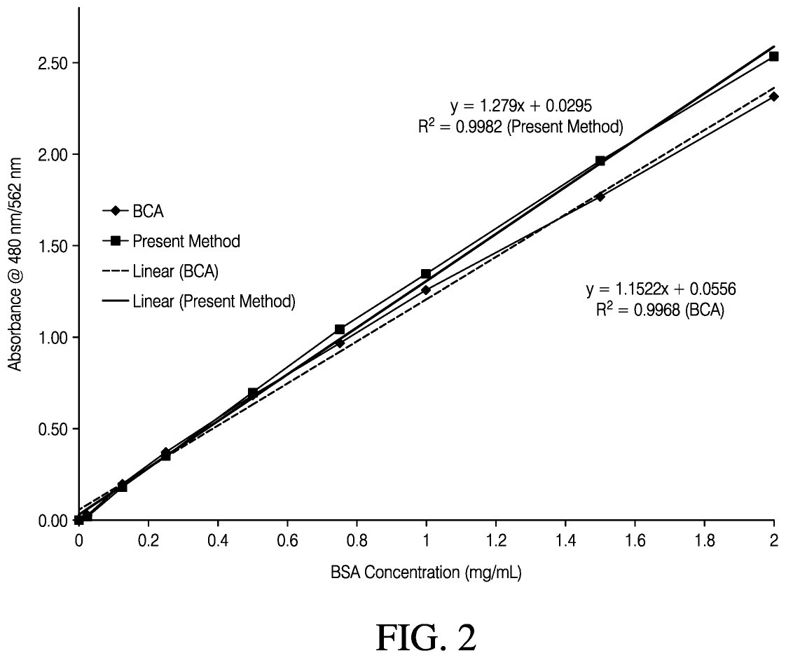

FIG. 2. depicts the calibration curve obtained for BSA using BCA and the Present Methods, which are referred to herein as current methods. The data shows a linear curve with increasing concentrations of BSA for both assays. However, in FIG. 2, the curve for the Present Methods is obtained within 5 minutes at Room Temperature. In comparison, the traditional BCA™ method required an incubation of 30 minutes at 37° C. to obtain a similar curve. Curves obtained from both assays are extremely linear with an R² of 0.99. The Present Methods and compositions provide a rapid assay compared to the traditional BCA™ assay used in the art.

### Example 3

### Lysate Protein Concentration Determination by the Present Methods & Present Compositions Compared with Traditional BCA™ & BCA™ Compositions

Unknown concentrations of lysates were determined by both traditional BCA™ and an exemplary Present Method using an exemplary current composition. The conditions such as sample volume, incubation time and temperature, absorbances used to read the plate are identical to those described in Table 4. The concentration of lysate proteins was determined using a BSA calibration curve in both the methods.

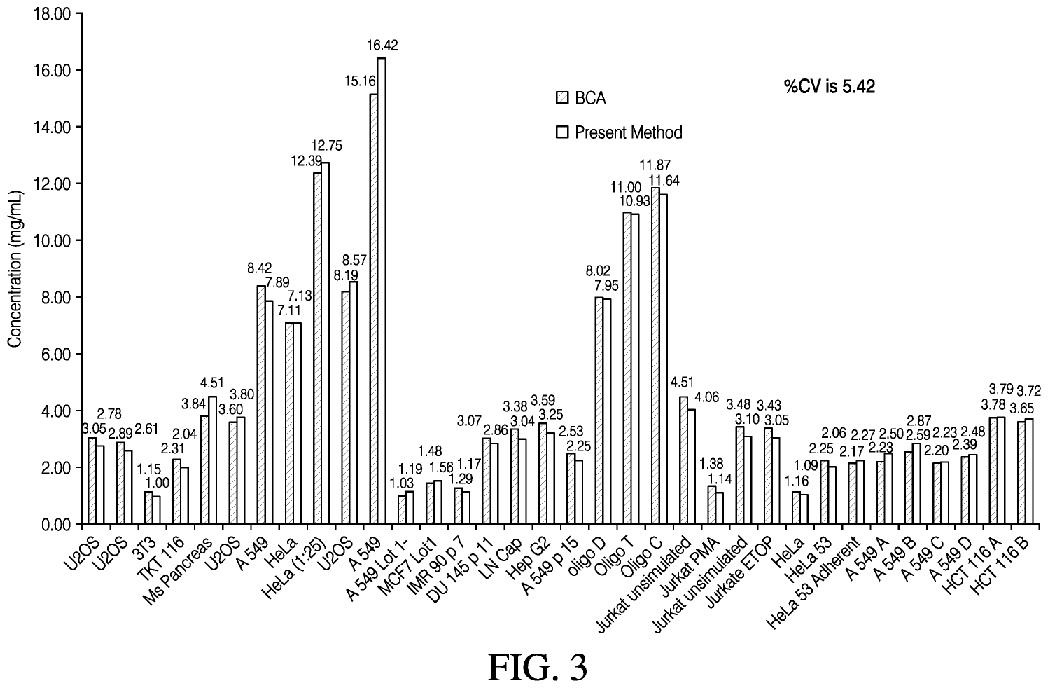

FIG. 3 shows similarities in concentration that was obtained for 34 different lysates using both assays, the traditional BCA™ assay described in Example 2 and an exemplary Present Method using an example of presently described compositions. Each lysate protein concentration was calculated using BSA as a standard and assayed using Traditional BCA™ and Present Method as shown in FIG. 3 using the conditions as described in Table 4. Very similar concentrations for unknown samples were obtained using Present Methods in just 5 minutes at RT. In contrast, it takes the traditional BCA™ method, 30 minutes at 37° C., to get similar results. The average % CV between the concentrations obtained using the assays were 5.4%. A paired t-test gave a p value of 0.675 (>0.05), which means there is statistically no difference between the concentrations obtained from the Present Methods and the BCA™ methods.

### Example 4

### Accuracy of Assays by Measuring Concentration of Known Protein Mixes

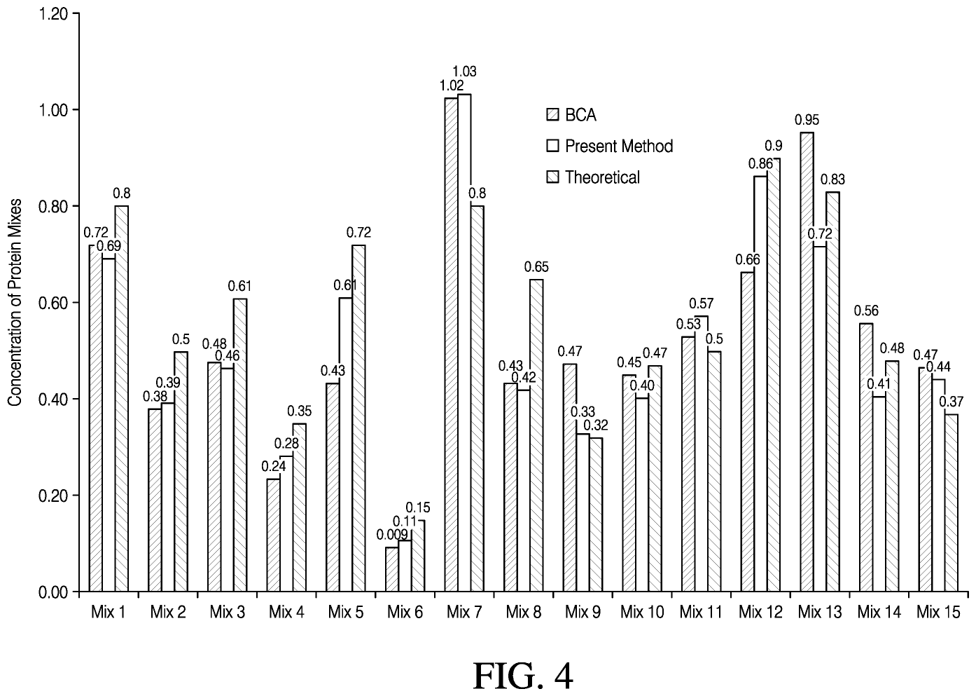

Protein mixes of known concentrations were made from commercially available proteins. The concentrations of the proteins with known extinction coefficients were determined using absorbance at 280 nm (known as Theoretical protein concentration determination). These proteins were then mixed in various ratios to generate a protein mixture with known concentration based on absorbance values at 280 nm. The concentrations of these protein mixes were determined by both traditional BCA™ and Present Methods using conditions identical to those described in Table 4. The concentrations of the protein mixtures determined using both the BCA™ and an exemplary Present Method were then compared to the Theoretical Protein Concentration (which were determined at absorbance at 280 nm) to determine how close each assays protein concentration is to the theoretical value. The results are depicted in FIG. 4.

FIG. 4. depicts comparison to the protein concentrations determinations obtained by the traditional BCA™ assay and an exemplary Current Method as compared further to Theoretical Protein Concentration Calculations. The purple bar represents concentrations obtained using the traditional BCA™ assay, the orange bar represents concentrations obtained using Present Methods and the grey bar is the Theoretical Protein Concentrations of the protein mixtures based on their absorbance at 280 nm. Data from FIG. 4 shows that the concentration obtained by both the assays is close to the theoretical concentration of the protein mixes. For the BCA™ assay a % CV of 18.6 was obtained with respect to the theoretical concentrations. For the Present Methods a % CV of 13.7 was obtained with respect to the theoretical concentrations.

### Example 5

### Present Methods and Compositions Provide Rapid Results

Exemplary Current Formulations used in an exemplary Present Method at varying times from 1 minute to 5 minutes at Room Temperature were compared to the traditional BCA™ Assay, which required 30 minutes and incubation at 37° C.

BSA standards at various concentrations starting from 2 mg/mL down to 0.125 mg/mL were used to generate a calibration curve for the Present Methods by incubating the working reagent, with the BSA standards at different times. The working reagent for both the traditional BCA™ and the presently disclosed methods was made by adding 50 parts of Reagent A to 1 part of Reagent B from the Tables 1 and 3 for each method. See Table 1 for exemplary compositions for Reagent A and Reagent B of the present method. See Table 3 for components of Reagent A and Reagent B of the traditional BCA™ method. The assays were performed on a microplate. The standards were added in triplicates. The following conditions were used for the assay is described in Table 5.

TABLE 5
Conditions and Parameters Used for Present Methods & Compositions

| | Present Method | |
|---|---|---|
| Standards/Sample volume | 20 | μL |
| Working reagent volume | 200 | μL |
| Incubation temperature | Room Temp | |
| Incubation time | 1 minute, 2 minutes, 3 minutes, 4 | |
| | minutes, and 5 minutes | |
| Absorbance at which plate | 480 | nm |
| was read | | |

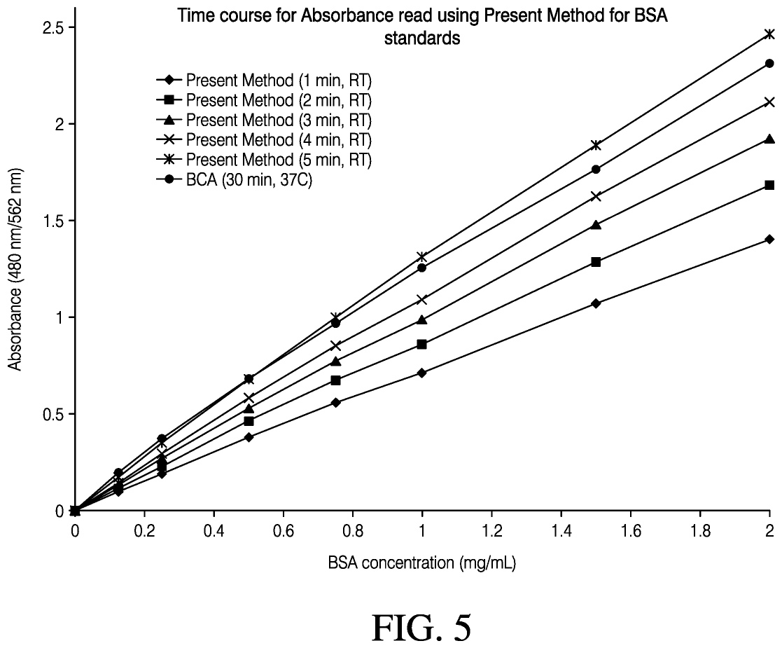

FIG. 5 depicts a BSA calibration curve obtained for an exemplary Present Method as described above at different time points starting from 1 minute to 5 minutes at Room Temperature. The data shows a linear curve with increasing slope as the incubation time increases. The curve using traditional BCA™ was also generated as a control, at 37° C., 30 minute incubation as a comparison to the Present Methods. Note: The sample volume used to generate the above data using Present Methods (20 μL) is lesser than the volume used for traditional BCA (25 μL).

### Example 6

### Comparison of Present Method and Traditional BCA™ Under Identical Incubation Conditions of Time and Temperature

BSA standards at various concentrations starting from 2 mg/mL down to 0.125 mg/mL were used to generate a calibration curve for an exemplary Present Method and for the traditional BCA™ method.

The working reagent for both the traditional BCA™ and the presently disclosed methods was made by adding 50 parts of Reagent A to 1 part of Reagent B from the Tables 1 and 3 for each method. See Table 1 for exemplary compositions for Reagent A and Reagent B of the present method. See Table 3 for components of Reagent A and Reagent B of the traditional BCA™ method.

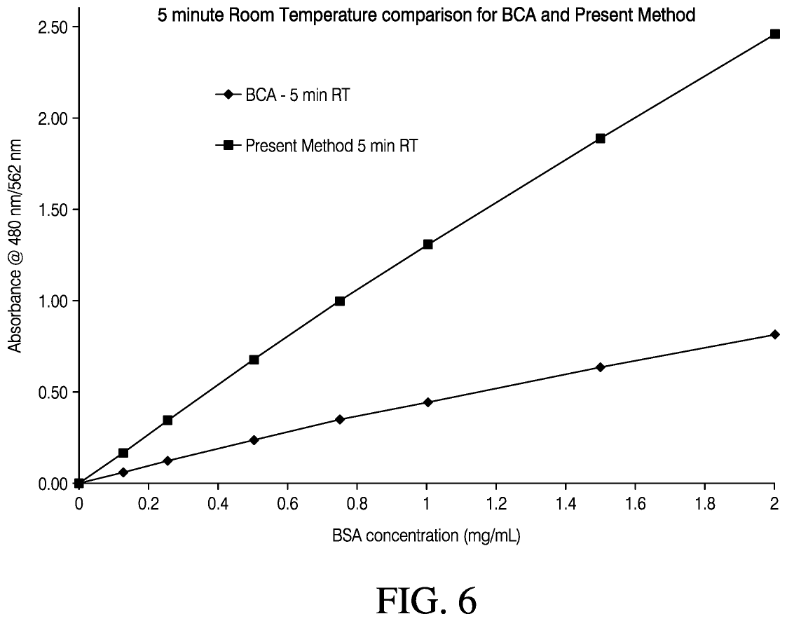

FIG. 6 shows a curve that was generated by using the same incubation time and temperature for both the assays which is 5 minutes at Room Temperature. FIG. 6 shows that at similar conditions of incubation time and temperature greater than 70% more signal is obtained using the Present Methods as compared to the traditional BCA™ method.

### Example 7

### Effect of Acetonitrile Concentration

Different exemplary compositions and formulations of the present disclosure, with varying concentration of acetonitrile, were tested. Two different exemplary compositions of the present disclosure, referred to herein as “buffer system A” and “buffer system B” due to the use of different buffers sodium carbonate or CAPS buffer, were each prepared with varying acetonitrile concentrations as described below to test the efficacy of some exemplary compositions of the present disclosure. A total of six different exemplary compositions of the present disclosure were tested, i.e., three exemplary compositions of Buffer A with 0%, 10% and 30% Acetonitrile respectively, & three exemplary compositions of Buffer B with 0%, 10% and 25% Acetonitrile respectively, as shown below:
Buffer A: Sodium Carbonate (0.32M), Sodium Bicarbonate (0.11M), Sodium Tartrate (0.8 mg/mL), Acetonitrile (0%, 10%, 30%)
Buffer B: CAPS buffer (0.2M), Sodium Bicarbonate (0.2M), Sodium Tartrate (0.8 mg/mL), Acetonitrile (0%, 10%, 25%)

The above compositions were tested to see the effect of increasing acetonitrile on the absorbance at 480 nm by varying the concentration of BSA.

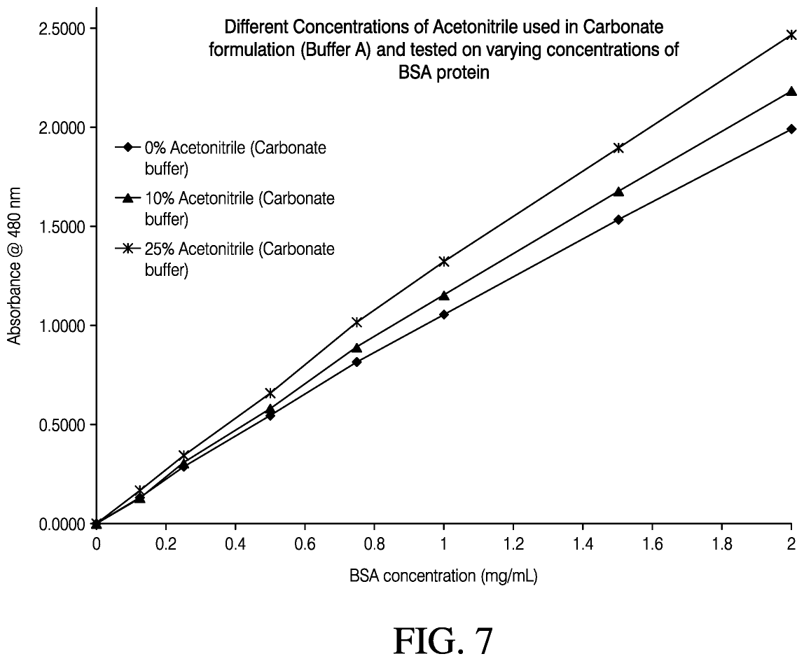

FIG. 7. shows the BSA calibration curve obtained for the Present Methods using Buffer A at the three different acetonitrile concentrations. BSA proteins at varying concentrations were added at 20 μL to a plate. The working reagent was prepared as per an exemplary Present Method protocol and was added at 200 μL/well. The plate was incubated for 5 minutes at Room Temperature. The Absorbance at 480 nm was plotted versus BSA concentration. The data shows that as acetonitrile concentration increases in the formulation the absorbance intensity also increases. There is an average of 22.3% increase of signal between the formulation that has 0% Acetonitrile to the formulation that has 25% Acetonitrile.

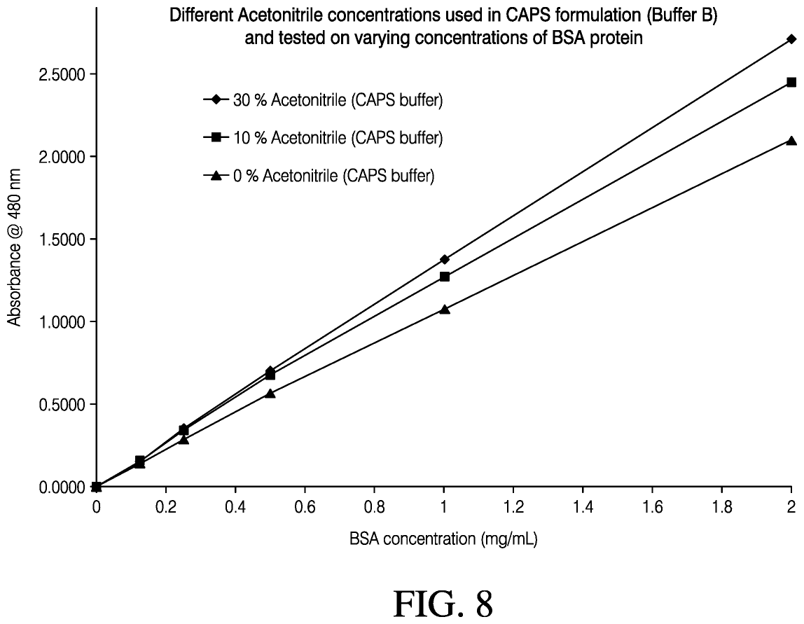

FIG. 8. shows the BSA calibration curve obtained for the Present Methods using Buffer B at three different acetonitrile concentrations. BSA proteins at varying concentrations were added at 20 μL to a plate. The working reagent was prepared as per an exemplary Present Method protocol and was added at 200 μL/well. The plate was incubated for 5 minutes at Room Temperature. The Absorbance at 480 nm was plotted versus BSA concentration. The data shows that as acetonitrile concentration increases in the formulation the absorbance intensity also increases. There is an average of 22.4% increase of signal between the formulation that has 0% Acetonitrile to the formulation that has 30% Acetonitrile.

### Example 8

### Fluorescence vs Absorbance Detection Modes of the Present Methods

BSA standards of different concentrations from 1 mg/mL to 0.125 mg/mL were used to generate a calibration curve. The working reagent for an exemplary Present Method was made by adding 50 parts of Reagent A to 1 part of Reagent B as described in Example 1. The Absorbance assay was performed on a microplate and the fluorescence assay was performed on Qubit 3.0 Fluorometer instrument.

TABLE 6
Conditions and parameters that were used for each assay

| | Absorbance assay | | Fluorescence assay | |
|---|---|---|---|---|
| | (Microplate) | | (Qubit) | |
| Standards/Sample | 20 | μL | 20 | μL |
| volume | | | | |
| Working reagent | 200 | μL | 200 | μL |
| volume | | | | |
| Incubation | Room Temp | | Room Temp | |
| temperature | | | | |
| Incubation time | 5 | minutes | 5 | minutes |

| | | | |
|---|---|---|---|
| Read out | 480 | nm | Fluorescence, Excitation |

| | |
|---|---|
| Absorbance | in Blue Channel at 470 |
| | nm and Emission in Red |
| | Channel at 665-720 nm |

Six protein mixes of known concentrations were made from commercially available proteins. The concentrations of the proteins with known extinction coefficients were determined using Absorbance at 280 nm. These proteins were then mixed in various ratios to get a protein mixture with known concentration (Actual or Theoretical concentration). The concentration of these protein mixes was determined using Present Methods using both Absorbance and Fluorescence mode using conditions, as described in Table 6. The concentration of protein mixes from both assays was determined using standard curve that is generated using BSA standards from both the modes. The concentrations thus obtained were then compared to determine how close they are to each other.

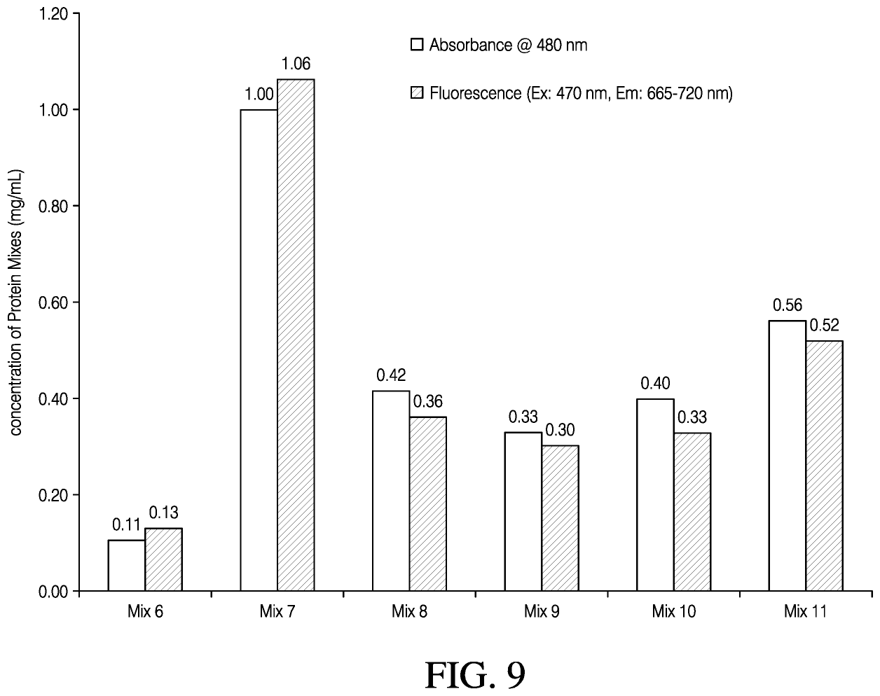

FIG. 9 shows comparison to the concentrations obtained by the two modes. The orange bar represents concentrations obtained using Absorbance mode, the red bar represents concentrations obtained using Fluorescence mode. The data above shows that the concentrations obtained by both the modes are close to each other. A p value for the paired t-test of 0.326 (>0.05) was obtained which indicates that the two values are not statistically significantly different from each other. Accordingly, according to one embodiment, the present disclosure provides compositions and methods that are amenable to being assayed on two different instrument platforms, fluorometer and spectrophotometer, with equal efficacy.

### Example 9

### Compositions & Methods for Rapid Methods Using Fluorometric Detection

Exemplary compositions of the disclosure were prepared as described below and tested according to an exemplary method of the disclosure comprising the steps of: (a) combining a sample with acetonitrile; and a reagent having the general formula (I) and copper to form a mixture; (b) incubating the mixture under conditions sufficient to form a colored complex; and (c) measuring the fluorescence change by excitation of the colored complex at a first wavelength and measuring emission at a second wavelength, wherein the fluorescence measured is indicator of protein or peptide concentration in the sample.

Exemplary compositions of the disclosure were tested by incubating the mixture at Room Temperature for 5 minutes and measuring fluorometrically. An exemplary composition, referred to as working reagent, was made by adding 49 parts of Reagent A and 1 part of Reagent B, as described in Example 1. The reactions were performed according to the conditions outlined in Table 7 and the fluorescence was read on the Qubit™ fluorometer instrument.

TABLE 7
Conditions and Parameters Used

| | Fluorescence assay (Qubit ™) | |
|---|---|---|
| BSA (1 mg/mL) | 20 | μL |
| Volume of Working reagent | 200 | μL |
| Incubation temperature | Room Temp | |
| Incubation time | 5 | minutes |
| Stop solution | 50 | μL |

| | |
|---|---|
| Read out | Fluorescence, Excitation in Blue Channel |
| | at 470 nm and Emission in Red Channel |
| | at 665-720 nm OR |
| | Fluorescence, Excitation in Blue Channel |
| | at 470 nm and Emission in Green |
| | Channel at 510 to 580 |

Fluorescence signal was generated by mixing BSA at a concentration of 1 mg/mL with the present method's working reagent and the assay was performed using the parameters in Table 7. The reactions were then stopped after 5 minutes of incubation with the addition of 50 μL of a stop solution that contained either 1M Hydrochloric acid, 0.16M Sulfuric acid, 0.1M Glycine pH 2.0, 0.1M Glycine pH 2.8, or water. The emission fluorescence was monitored in both the Green and Red spectra and was monitored at various time points over the course of 1 hour.

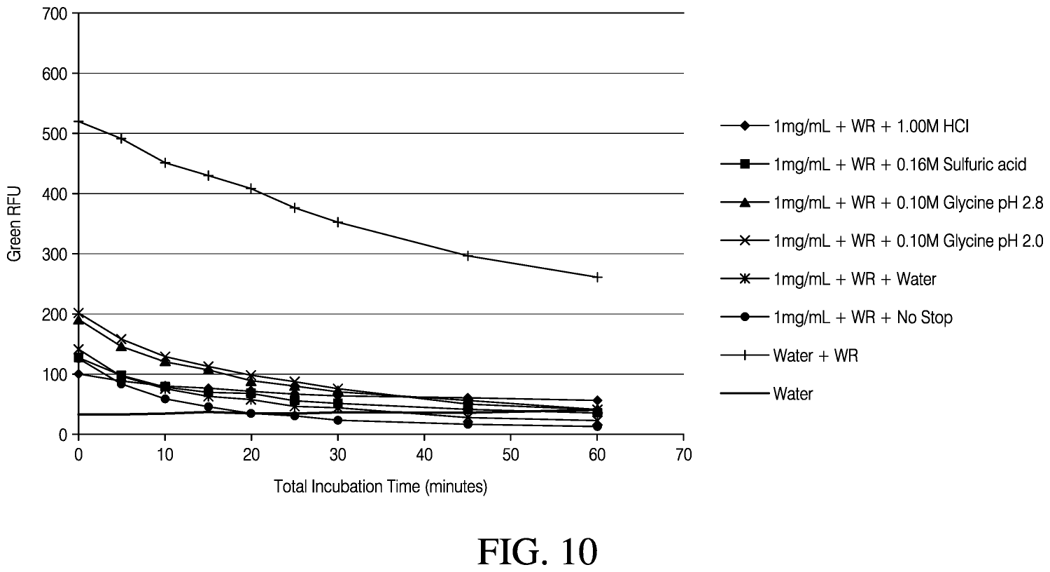

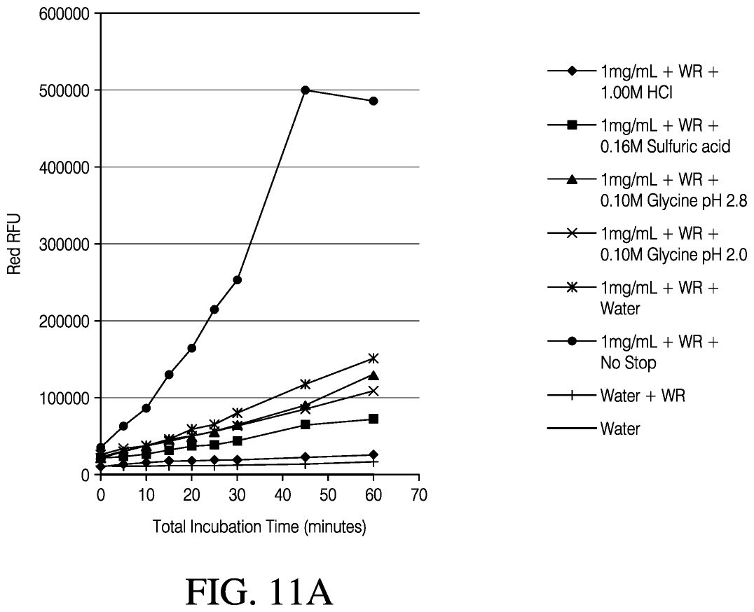

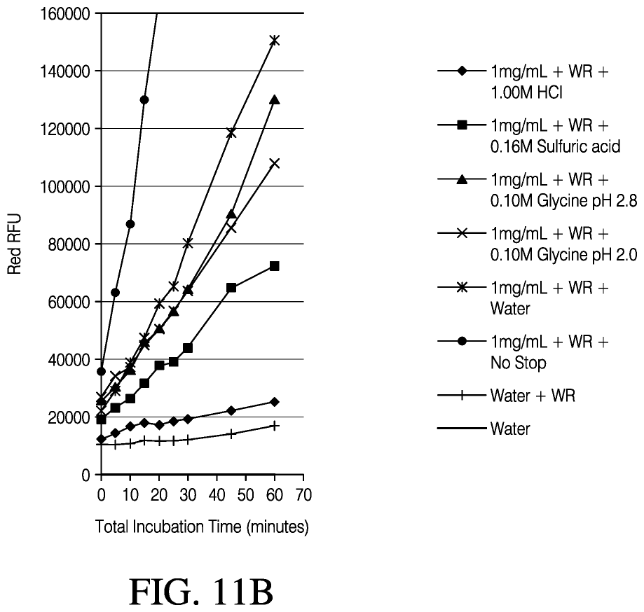

FIG. 10, FIG. 11A and FIG. 11B depict graphs that compare the stop effectiveness of several stop solutions for the assay, results were read for both the Green (A) and Red (B) spectra as detected by the Qubit instrument. All stop solutions used herein prevented increase in signal, from the fluorescence emissions, to signal levels that were above the detectable range of the detection instruments. Using stop solutions provided a more efficient way to measure fluorescence as compared to samples that were untreated or treated with water. Hydrochloric showed the best inhibition of signal in both the Red spectra and Green spectra.

In some embodiments, the current methods are not endpoint assays. As long as there is still Cu²⁺ present in the reaction mixture which has not been reduced from Cu²⁺ to Cu¹⁺, sample protein will continue to reduce the Cu²⁺ until all Cu²⁺ in the reaction mix is exhausted. In view of this, if the reaction mix is allowed to sit for a long time, the signal will continue to increase over time. Using a stop solution in the reaction mixture, at a fixed time such as at 5 minutes as in the present Example, or any time chosen by the experimenter that the Present Methods allow (such as less than 5 minutes, i.e. 5, 4, 3, 2 or 1 minutes or less than 1 minute), which time is sufficient to measure the sample protein concentration as demonstrated herein, prevents increase of signal as described above.

Furthermore, as the present methods are extremely fast, if one has multiple samples to test (for example 15 samples) and one is reading samples in a single tube format, as in the currently used Qubit™ platform, sample #1 could incubate for 5 min but sample #15 could incubate for more than 5 minutes before it is read by the experimenter. To avoid having to time the assay perfectly, one could incubate all samples for 5 min (or any other time as preferred by the experimenter that is 5 minutes or less than 5 minutes) and then simultaneously stop the assay by addition of a stop solution as described herein which include any one of hydrochloric acid, sulphuric acid (as described in this example) or acetic acid or formic acid or citric acid, etc. described in Examples below) to prevent assay drift due to increase in signal in one sample versus the other that will be compared for protein or peptide concentration determination.

Furthermore, if the standard protein/peptide curve samples are read in an exemplary time of 5 minutes (or other time as chosen by experimenter), the test samples must also be read at the same incubation time of 5 minutes otherwise the test samples will show artificially high protein concentration values. Using a stop solution as described in this disclosure at a fixed incubation time for both the standard sample and the test sample allows for accurate results.

### Example 10

### Enhancement of Signal Using Metal Chelator

The working reagent as described in Example 1 was made by adding 49 parts of Reagent A and 1 part of Reagent B. Reactions were performed according to the conditions outlined in Table 8 and the fluorescence was read on the Qubit instrument.

TABLE 8
Conditions and Parameters Used for the Assay

| | Fluorescence assay (Qubit) | |
|---|---|---|
| BSA (1 mg/mL) | 20 | μL |
| Working reagent volume | 200 | μL |
| Incubation temperature | Room Temp | |
| Incubation time | 5 | minutes |
| Stop solution containing | 50 | μL |
| an Enhancer | | |

| | |
|---|---|
| Read out | Fluorescence, Excitation in Blue Channel |
| | at 470 nm and Emission in Red Channel |
| | at 665-720 nm |

Metal chelators such as Nitrilotriacetic acid (NTA), Ethylenediamine tetraacetic acid (EDTA), Iminodiacetic acid (IDA), Triscarboxymethyl ethylene diamine (TED) can be used as signal enhancing agents. Of these NTA and EDTA were tested in the present Example.

Fluorescence signal was generated by mixing BSA at a concentration of 1 mg/mL with the present method's working reagent and the assay was performed using the parameters in Table 8. After 5 minutes of incubation a 50 μL of a stop solution was added that contained either 10 mM EDTA, 1 mM EDTA, 10 mM NTA or 1 mM NTA. The Red emission fluorescence was monitored at various time points over the course of 1 hour.

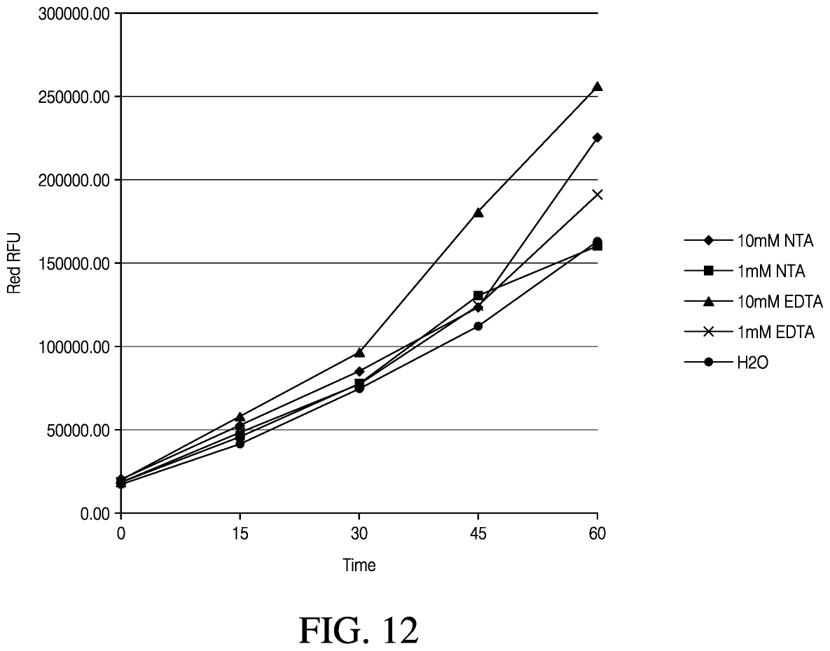

FIG. 12 depicts graphs that compare the effectiveness of several stop solutions for the assay. None of the solution prevented signal increase, but instead enhanced the signal output. EDTA offered some degree of enhancement of signal at both concentrations while NTA only offered signal enhancement at the higher concentration.

### Example 11

### Alternative Concentrations for Acid Stop

Stop solutions for fluorescence measurement: The working reagent was made by adding 49 parts of Reagent A and 1 part of Reagent B as described in Example 1. The reactions were performed according to the conditions outlined in Table 1 and the fluorescence was read on the Qubit instrument.

TABLE 9
Conditions and parameters that were used for the assay

| | Fluorescence assay (Qubit) | |
|---|---|---|
| BSA (1 mg/mL) | 20 | μL |
| Working reagent volume | 200 | μL |
| Incubation temperature | Room Temp | |
| Incubation time | 5 | minutes |
| Stop solution | 50 | μL |

| | |
|---|---|
| Read out | Fluorescence, Excitation in Blue Channel |
| | at 470 nm and Emission in Red Channel |
| | at 665-720 nm |

Fluorescence signal was generated by mixing BSA at a concentration of 1 mg/mL with the present method's working reagent and the assay was performed using the parameters in Table 9. The reactions were then stopped after 5 minutes of incubation with the addition of 50 μL of a stop solution that contained either 1, 2, 4, or 6M Hydrochloric acid or water. The emission fluorescence was monitored in the Red spectra and was monitored at various time points over the course of 1 hour.

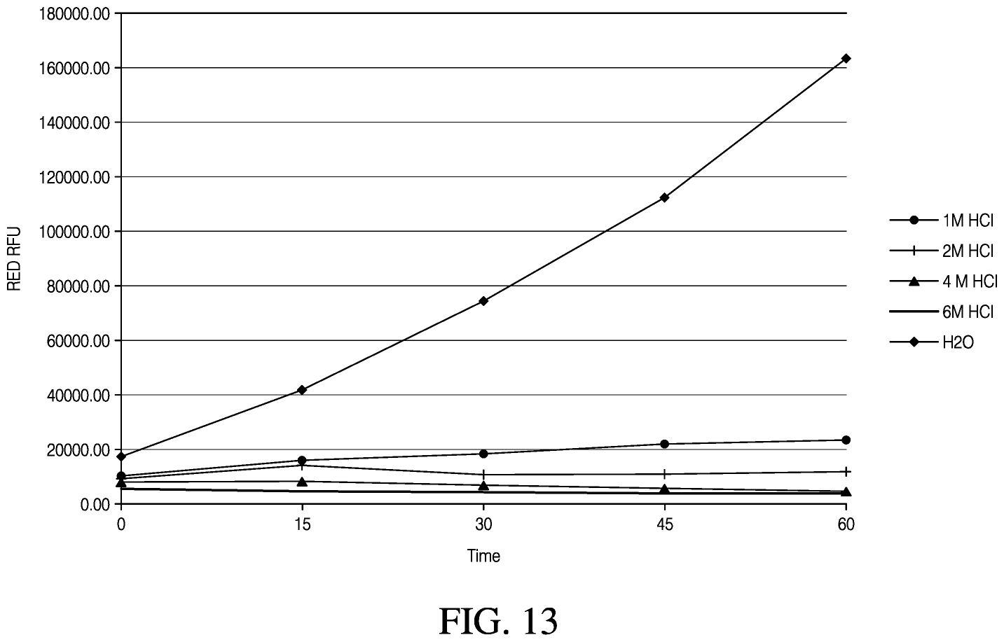

The graph in FIG. 13 compares the effectiveness of several hydrochloric acid stop solution. In all reactions, an inhibition of the fluorescence signal was observed and at the highest concentrations (4 and 6M) a decrease in signal was observed.

### Example 12

### Optimization of Concentration and Volume of Stop Solution

An exemplary working reagent for the present method was made by adding 49 parts of Reagent A and 1 part of Reagent B as described in Example 1. The reactions were performed according to the conditions outlined in Table 10 and the fluorescence was read on the Qubit instrument.

TABLE 10
Conditions and parameters that were used for the assay

| | Fluorescence assay (Qubit) | |
|---|---|---|
| BSA (1 mg/mL) | 20 | μL |
| Working reagent volume | 200 | μL |
| Incubation temperature | Room Temp | |
| Incubation time | 5 | minutes |
| Stop solution | 50-200 | μL |

| | |
|---|---|
| Read out | Fluorescence, Excitation in Blue Channel |
| | at 470 nm and Emission in Red Channel |
| | at 665-720 nm |

Fluorescence signal was generated by mixing BSA at a concentration of 1 mg/mL with the working reagent as described in Example 1 and the assay was performed using the parameters in Table 10. The reactions were then stopped after 5 minutes of incubation with the addition of 50, 100, or 200 μL of a stop solution that contained either 3M Hydrochloric acid, 2M Sulfuric acid, or 1M Sulfuric acid. The emission fluorescence was monitored in the Red spectra and was monitored at various time points over the course of 1 hour.

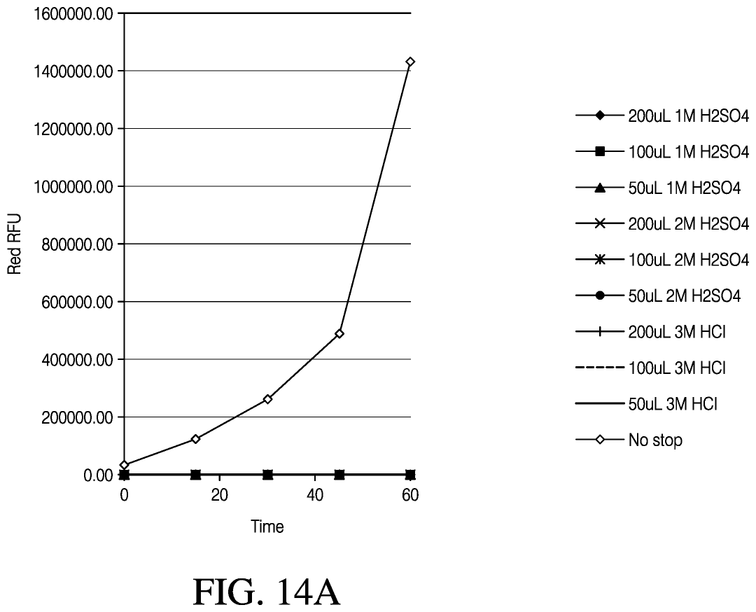

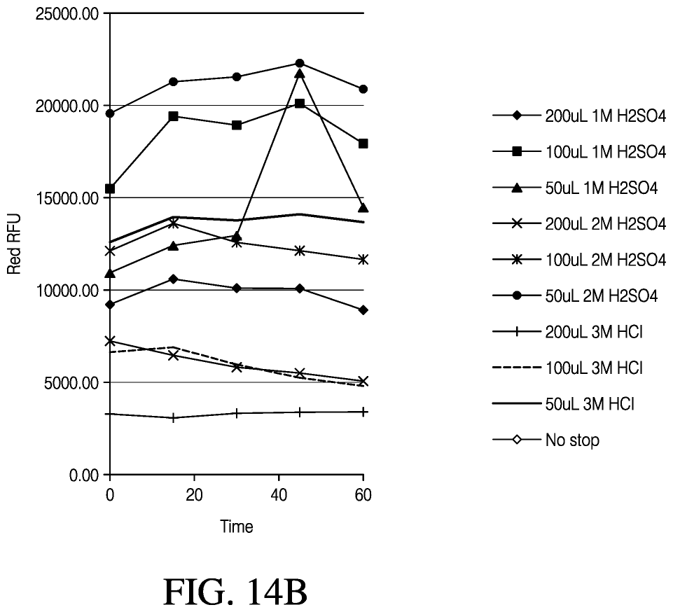

FIGS. 14A and 14B show a comparison of multiple acid concentrations and volumes. The results show that an effective stop can be achieved with multiple acidic solutions. The ability to stop the reaction depends on the type of acid and total amount of acid added to the reaction. For example, the reaction shows a similar change in signal over the course of 60 min upon the addition of 100 μL of a 2M sulfuric acid solution or 200 μL of a 1M sulfuric acid solution

### Example 13

### Acetic Acid as a Stop Solution

An exemplary working reagent was made by adding 49 parts of Reagent A to 1 part of Reagent B as described in Example 1. The reactions were performed according to the conditions outlined in Table 11 and the fluorescence was read on the Qubit instrument.

TABLE 11
Conditions and parameters that were used for the assay

| | Fluorescence assay (Qubit) | |
|---|---|---|
| BSA (1 mg/mL) | 20 | μL |
| Working reagent volume | 200 | μL |
| Incubation temperature | Room Temp | |
| Incubation time | 5 | minutes |
| Stop solution | 100 | μL |

| | |
|---|---|
| Read out | Fluorescence, Excitation in Blue Channel |
| | at 470 nm and Emission in Red Channel |
| | at 665-720 nm |

Fluorescence signal was generated by mixing BSA at a concentration of 1 mg/mL with the working reagent and the assay was performed using the parameters in Table 11. The reactions were then stopped after 5 minutes of incubation with the addition of 100 μL of a stop solution that contained either 8M urea, 6 guanidine, 0.5M sodium meta-periodate, 2M sodium hydroxide, or 2M acetic acid. The emission fluorescence was monitored in the Red spectra and was monitored at various time points over the course of 1 hour.

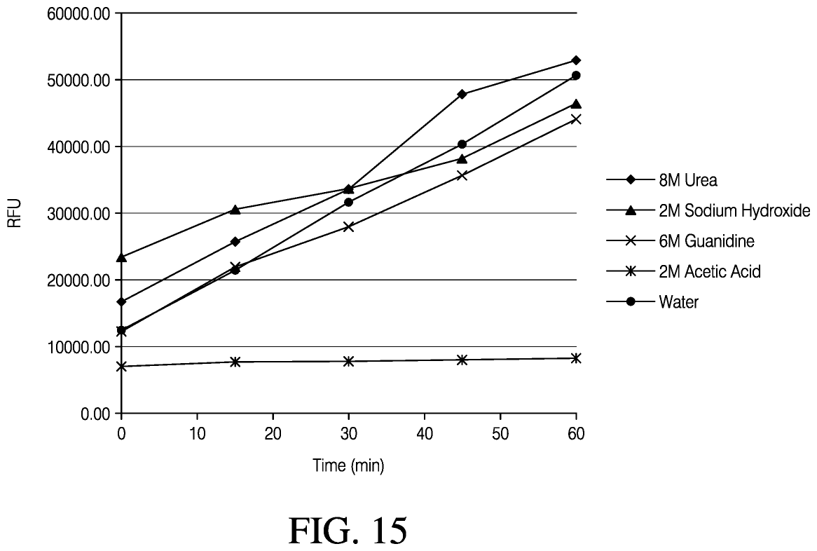

The graph in FIG. 15 compares the different effectiveness of several stop solutions. The graph shows that the only stop solution that worked among those tested is the acetic acid solution.

### Example 14

### Acetic Acid, Formic Acid and Citric Acid as a Stop Solutions

An exemplary working reagent for the present method was made by adding 49 parts of Reagent A and 1 part of Reagent B as described in Example 1. The reactions were performed according to the conditions outlined in Table 12 and the fluorescence was read on the Qubit instrument.

TABLE 12
Conditions and parameters that were used for the assay

| | Fluorescence assay (Qubit) | |
|---|---|---|
| BSA (1 mg/mL) | 20 | μL |
| Working reagent volume | 100 | μL |
| Incubation temperature | Room Temp | |
| Incubation time | 5 | minutes |
| Stop solution | 100 | μL |

| | |
|---|---|
| Read out | Fluorescence, Excitation in Blue Channel |
| | at 470 nm and Emission in Red Channel |
| | at 665-720 nm |

Fluorescence signal was generated by mixing BSA at a concentration of 1 mg/mL with the present methods working reagent and the assay was performed using the parameters in Table 12. The reactions were then stopped after 5 minutes of incubation with the addition of 100 μL of a stop solution that contained either 2M formic, 2M citric, or 2M acetic acid. The emission fluorescence was monitored in the Red spectra and was monitored at various time points over the course of 1 hour.

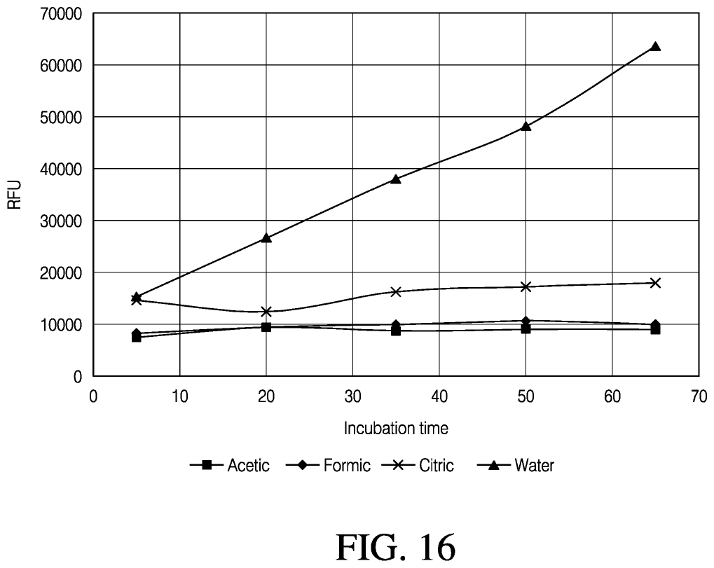

The graph in FIG. 16 compares the different effectiveness of stop solutions of acetic acid, formic acid and citric acid. The graph shows that while all three solutions inhibit signal increase formic acid and acetic acid were comparatively more effective as a stop solution.

### Example 15

### Rapid Fluorescent Assay

The time line for initiation of fluorescence signal for a method of the present disclosure was analyzed. An exemplary working reagent for the Present Method was made by adding 49 parts of Reagent A to 1 part of Reagent B as described in Example 1. The reactions were performed according to the conditions outlined in Table 13 and the fluorescence was read on the Qubit instrument.

TABLE 13
Conditions and Parameters Used

| | Fluorescence Assay (Qubit) | |
|---|---|---|
| BSA (1 mg/mL) | 20 | μL |
| Working reagent volume | 200 | μL |

| | |
|---|---|
| Incubation temperature | Room Temp |
| Incubation time | 0 minutes to 5 minutes in 15 second |
| | increments |
| Read out | Fluorescence, Excitation in Blue Channel |
| | at 470 nm and Emission in Red Channel |
| | at 665-720 nm |

Fluorescence signal was generated by mixing BSA at a concentration of 1 mg/mL with the present methods working reagent and the assay was performed using the parameters in Table 13. The emission fluorescence was monitored in the Red spectra and was monitored from 0 minutes of incubation at every 15 second increment over the course of 5 minutes.

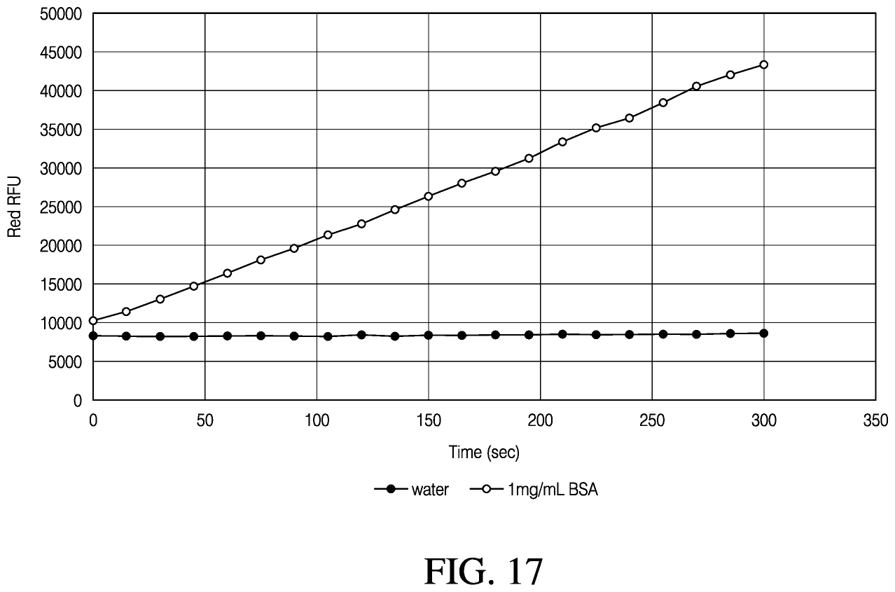

The graph of FIG. 17 shows the time line of start of the fluorescence signal detection by the fluorometer. As can be seen fluorescence signal was detected even as early as 15 seconds from the reaction. In embodiments of the present method, fluorescence signal from the reaction due to the presence of protein in a sample is immediately after mixing the working reagent with the protein and continues to increase through the end of the time course of 5 minutes. Accordingly, methods, compositions and kits of the method are effective in generating rapid protein/peptide concentration determination, i.e. instantaneous, in 15 seconds, in 30 seconds, in 45 seconds, in less than 1 minute, and in 1-5 minutes including time ranges in between.

### Example 15

### Additional Exemplary Compositions

Several exemplary compositions as described in the disclosure have been shows to be useful for protein/peptide quantification in exemplary methods described herein. In addition to some exemplary compositions described in the Examples above, additional exemplary compositions were tested using embodiments of present methods to determine protein concentrations. Tables 14, 15 and 16, outline 3 different exemplary compositions according to present embodiments.

TABLE 14
One Exemplary Composition

| Reagent A | Reagent B |
|---|---|
| 0.043 M Sodium Carbonate | 80 mg/mL Cupric Sulfate |
| 0.193 M Sodium Bicarbonate | |
| 0.8 mg/mL Sodium Tartrate | |
| 0.01 M Bathocuproine | |
| disulfonic acid | |
| 25% Acetonitrile | |
| pH 12.2 | |

TABLE 15
One Exemplary Composition

| Reagent A | Reagent B |
|---|---|
| 0.03 M Borate buffer | 80 mg/mL Cupric Sulfate |
| 0.2 M Sodium Bicarbonate | |
| 0.8 mg/mL Sodium Tartrate | |
| 0.01 M Bathocuproine | |
| disulfonic acid | |
| 10% Acetonitrile | |
| pH 11.85 | |

TABLE 16
One Exemplary Composition

| Reagent A | Reagent B |
|---|---|
| 0.03 M Borate buffer | 80 mg/mL Cupric Sulfate |
| 0.2 M Sodium Bicarbonate | |
| 0.8 mg/mL Sodium Tartrate | |
| 0.01 M Bathocuproine | |
| disulfonic acid | |
| 10% Acetonitrile | |
| pH 11.85 | |

BSA standard curve were generated using Traditional BCA™ method using BCA™ formulations as described in Table 3 above & for an exemplary Present Method using each of exemplary composition formulations described in Table 14, 15 and 16 respectively. BSA standards at various protein concentrations starting from 2 mg/mL down to 0.125 mg/mL were used to generate a calibration curve for both BCA™ and an exemplary Present Method.

The working reagents were made by adding 50 parts of Reagent A to 1 part of Reagent B as described in Table 3 for BCA™ Methods and as described in Tables 14, 15 and 16 for Present Methods. The assays were performed on a microplate. The conditions described in Table 17 were used for each assay.

TABLE 17
Conditions and Parameters that were Used for Each Assay

| | | Present Method (with |
|---|---|---|
| | BCA ™ | different formulations) |
| Standards/Sample volume | 25 μL | 20 μL or 25 μL |
| Working reagent volume | 200 μL | 200 μL |
| Incubation temperature | 37° C. | Room Temp |
| Incubation time | 30 minutes | 5 minutes |
| Absorbance at which plate | 562 nm | 480 nm |
| was read | | |

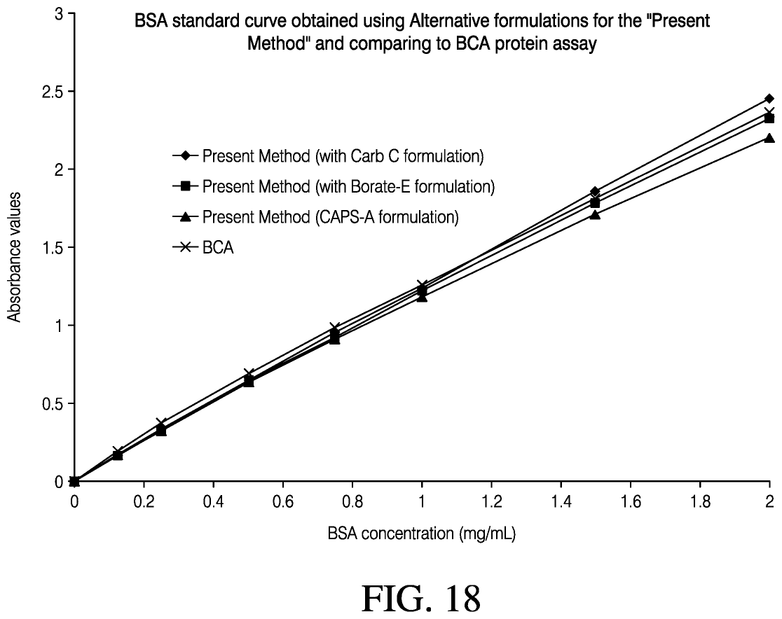

FIG. 18, depicts a calibration curve obtained for BSA standard using BCA™ Method and the three different formulations for the Present Method (described in FIG. 18 as Carb-C, Borate-E and CAPS-A). The data shows a linear curve with increasing concentrations of BSA for both assays.

The embodiments shown and described herein are only specific embodiments and are not limiting in any way. Therefore, various changes, modifications, or alterations to those embodiments may be made without departing from the spirit of the invention in the scope of the following claims. The references cited are expressly incorporated by reference herein in their entirety.

Each embodiment disclosed herein may be used or otherwise combined with any of the other embodiments disclosed. Any element of any embodiment may be used in any embodiment. Although the invention has been described with reference to specific embodiments, it will be understood by those skilled in the art that various changes may be made and equivalents may be substituted for elements thereof without departing from the true spirit and scope of the invention. In addition, modification may be made without departing from the essential teachings of the invention.

What is claimed is:

1. A method for determining protein or peptide concentration in a sample comprising:
(a) forming a mixture by combining the sample with:
(i) copper;
(ii) acetonitrile; and
(iii) a reagent comprising general formula (I) depicted below: 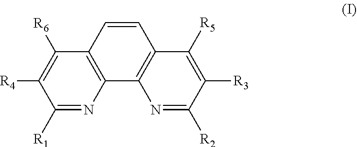
where,
each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅);
each of R₃, R₄, R₅ and R₆ is also additionally independently selected from the group consisting of hydrogen (H), sulfonate (—SO₃⁻) salt of sodium (Na⁺), sulfonate (—SO₃⁻) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺);
wherein when R₅ and R₆ is a phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and
wherein the reagent is present in a hydrated, ((I) H₂O), or a non-hydrated form;
(b) incubating the mixture under conditions sufficient to form a colored complex; and
(c) measuring the fluorescence change by excitation of the colored complex at a first wavelength and measuring emission at a second wavelength, wherein the fluorescence measured is an indicator of protein or peptide concentration in the sample OR by measuring absorbance of the colored complex at 450 nm to 500 nm as a direct indicator of protein or peptide concentration in the sample.

2. The method of claim 1, wherein the first wavelength is between 450 nm to about 480 nm.

3. The method of claim 1, wherein the second wavelength is between 660 nm to about 730 nm or is between 510 nm to about 580 nm.

4. The method of claim 1, wherein the fluorescence change is determined by a fluorometer and wherein the absorbance of the colored complex is measured by a spectrophotometer or an automated microplate reader.

5. The method of claim 1 further comprising, determining protein or peptide concentration in the sample by comparing the fluorescence or absorbance measured in step (c) with fluorescence or absorbance measured of at least one control standard sample containing a known concentration of a protein or peptide.

6. The method of claim 5, where the control standard sample is a protein, a peptide, a peptide mixture, or a protein digest.

7. The method of claim 1, wherein the copper provides a source of Cu²⁺ ions.

8. The method of claim 1, wherein the copper is comprised in copper (II) sulphate, copper (II) bromide, copper (II) chloride, copper (II) fluoride, copper (II) perchlorate, copper (II) molybdate, copper (II) nitrate, copper (II) hydroxide, copper (II) tetrafluoroborate,

9. The method of claim 1, wherein concentration of the acetonitrile is measured in volume/volume % is 5%, 10%, 15%, 20%, 25%, 30%.

10. The method of claim 1, where the sample is further combined with tartrate.

11. The method of claim 1, wherein the sample is further combined with sodium bicarbonate.

12. The method of claim 1, wherein the sample is further combined with a buffer selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

13. The method of claim 1, wherein the mixture has a pH range of from about 11-12.2.

14. The method of claim 1, where the incubating is at room temperature from about 18° C. to about 26° C.

15. The method of claim 1, wherein the colored complex forms and can be measured either colorimetrically or as a fluorescent emission in less than 25 minutes, less than 10 minutes, in 5 minutes, in less than 5 minutes, in 4 minutes, in 3 minutes, in 2 minutes, in 1 minute, in less than 1 minute, in 45 seconds, in 30 seconds, in 15 seconds or instantaneously.

16. The method of claim 1 wherein proteins or peptides can be detected in concentrations of from 20 μg/ml to 2000 μg/ml.

17. The method of claim 1 where the sample comprises a plurality of proteins or peptides.

18. The method of claim 1, wherein the reagent comprising general formula (I) comprises: 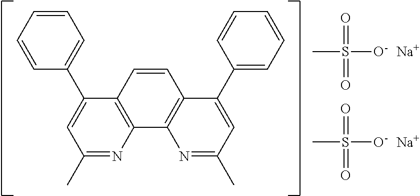
and is a hydrate or a non-hydrate form of the above.

19. The method of claim 1, wherein the reagent of general formula (I) comprises: 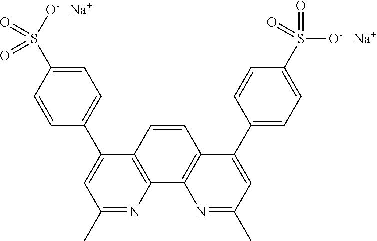
and is a hydrate or a non-hydrate form of the above.

20. The method of claim 1, wherein the reagent of general formula (I) comprises: 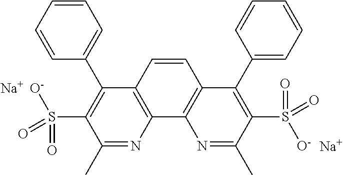
and is a hydrate or a non-hydrate form of the above.

21. The method of claim 1, wherein the reagent of general formula (I) comprises: 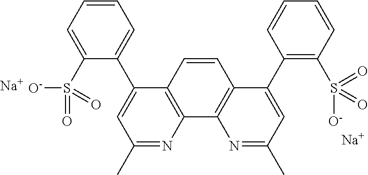
and is a hydrate or a non-hydrate form of the above.

22. The method of claim 1 where the sample is in an aqueous solvent, an organic solvent, and combinations thereof.

23. The method of claim 1 further comprising analyzing the proteins or peptide(s) by one or more method including chromatography, electrophoresis, immunoassays, mass spectrometry, nuclear magnetic resonance (NMR), or infrared spectroscopy (IR).

24. The method of claim 1 where the sample comprises at least one of an organic solvent, a detergent, a reagent to improve protein or peptide solubility or stability.

25. The method of claim 24, wherein the detergent is one or more of Triton X-100, Triton X-114, NP-40, Tween 80, Tween 20, CHAPS, and SDS.

26. The method of claim 1, further comprising adding a stop solution after step (b) and prior to step (c).

27. The method of claim 26, wherein the stop solution comprises one or more of acetic acid, citric acid, formic acid, hydrochloric acid, or sulphuric acid.

28. The method of claim 1, further comprising adding an enhancer after step (b) and prior to step (c).

29. The method of claim 28, wherein the enhancer comprises a metal chelator, Nitrilotriacetic acid (NTA), Ethylenediamine tetraacetic acid (EDTA), Iminodiacetic acid (IDA) or triscarboxymethyl ethylene diamine (TED).

30. The method of claim 1, further comprising adding a stop solution and an enhancer after step (b) and prior to step (c).

31. A composition comprising:
acetonitrile; and
a reagent having general formula (I): 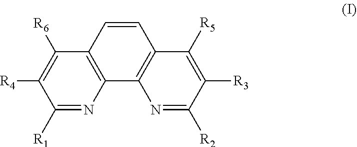
where,
each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅);
each of R₃, R₄, R₅ and R₆ is also additionally independently selected from the group consisting of hydrogen (H), sulfonate (—SO₃⁻) salt of sodium (Na⁺), sulfonate (—SO₃) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂) salt of potassium (K⁺), and carboxylate (—CO₂⁻) salt of lithium (Li⁺);
wherein when R₅ and R₆ is a phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and
wherein the reagent is present in a hydrated, ((I) H₂O), or a non-hydrated form.

32. The composition of claim 31, wherein the reagent of general formula (I) is selected from the group consisting of: 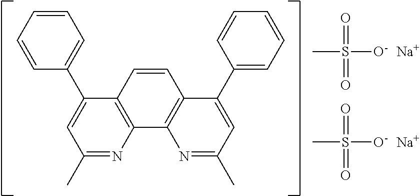
and a hydrate or a non-hydrate form of the above, 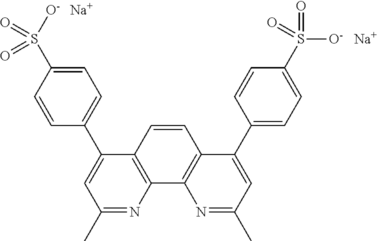 [SMILES: Cc1cc(-c2ccc(S(=O)(=O)[O-])cc2)c2ccc3c(-c4ccc(S(=O)(=O)[O-])cc4)cc(C)nc3c2n1 ; [Na+] ; [Na+]]
and a hydrate or a non-hydrate form of the above, 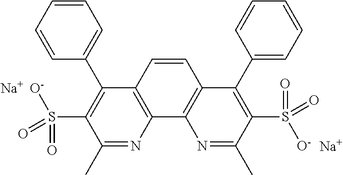 [SMILES: [Na+] ; Cc1nc2c(ccc3c(-c4ccccc4)c(S(=O)(=O)[O-])c(C)nc32)c(-c2ccccc2)c1S(=O)(=O)[O-] ; [Na+]]
and a hydrate or a non-hydrate form of the above, and 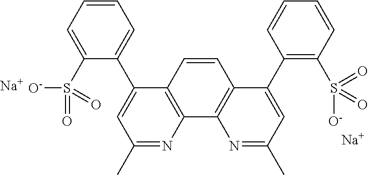 [SMILES: [Na+] ; Cc1cc(-c2ccccc2S(=O)(=O)[O-])c2ccc3c(-c4ccccc4S(=O)(=O)[O-])cc(C)nc3c2n1 ; [Na+]]
and a hydrate or a non-hydrate form of the above.

33. The composition of claim 31, wherein concentration the reagent of formula (I) is from about 0.01 M to 0.1 M; and concentration of acetonitrile is from about 5%-50%.

34. The composition of claim 31, further comprising a tartrate at a concentration ranging from about 5.7 mM to about 22.7 mM.

35. The composition of claim 34, wherein the tartarate is sodium tartarate, potassium tartrate, sodium potassium tartrate.

36. The composition of claim 31, further comprising sodium bicarbonate or potassium bicarbonate.

37. The composition of claim 31, further comprising a buffer selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

38. The composition of claim 31, further comprising copper.

39. The composition of claim 38, wherein the copper is comprised in copper (II) sulphate, copper (II) bromide, copper (II) chloride, copper (II) fluoride, copper (II) perchlorate, copper (II) molybdate, copper (II) nitrate, copper (II) hydroxide, copper (II) tetrafluoroborate.

40. The composition of claim 31, wherein concentration of copper is from about 0.25 mM to about 0.5 mM.

41. The composition of claim 31, wherein the pH is from about 11-12.2.

42. A kit comprising:
1) a composition comprising acetonitrile and a reagent having the general formula (I): 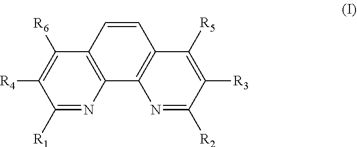
where,
each of R₁, R₂, R₃, R₄, R₅ and R₆ is independently alkyl including but not limited to a C₁-C₆ straight or branched alkyl or a C₆-C₂₀ aryl, alkylaryl, or arylalkyl such as methyl (—CH₃), ethyl (—CH₂CH₃), propyl (—CH₂CH₂CH₃), butyl (—CH₂CH₂CH₂CH₃) or phenyl (—C₆H₅);
each of R₃, R₄, R₅ and R₆ is also additionally independently selected from the group consisting of hydrogen (H), sulfonate (—SO₃⁻) salt of sodium (Na⁺), sulfonate (—SO₃) salt of potassium (K⁺), sulfonate (—SO₃⁻) salt of lithium (Li⁺), phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺), and carboxylate (—CO₂⁻) salt of lithium (Li⁺);
wherein when R₅ and R₆ is a phenyl (—C₆H₅), the phenyl (—C₆H₅) can additionally independently have attached to it a molecule selected from the group consisting of sulfonate (—SO₃⁻) salt of sodium (Na⁺), sulfonate (—SO₃⁻) salt of potassium (K⁺), sulfonate (—SO₃⁻) salt of lithium (Li⁺); phosphonate (—PO₃⁻) salt of sodium (Na⁺), phosphonate (—PO₃⁻) salt of potassium (K⁺), phosphonate (—PO₃⁻) salt of lithium (Li⁺), carboxylate (—CO₂⁻) salt of sodium (Na⁺), carboxylate (—CO₂⁻) salt of potassium (K⁺) and carboxylate (—CO₂⁻) salt of lithium (Li⁺); and
wherein the reagent is present in a hydrated, ((I) H₂O), or a non-hydrated form; and
2) copper;
each ingredient contained in one or more separate containers.

43. The kit of claim 42, wherein the composition further comprises one or more ingredients including a tartarate selected from the group consisting of sodium tartarate, potassium tartarate, sodium bicarbonate, potassium bicarbonate, sodium potassium bicarbonate, and one or more buffers selected from the group consisting of 3-(Cyclohexylamino)-1-propanesulfonic acid (CAPS), Borate, Carbonate-Bicarbonate, 4-(Cyclohexylamino)-1-butanesulfonic acid (CABS), 3-(Cyclohexylamino)-2-hydroxy-1-propanesulfonic acid (CAPSO), N-tris(Hydroxymethyl)methyl-4-aminobutanesulfonic acid (TABS) 4-(N-Morpholino)butanesulfonic acid (MOBS) 2-(Cyclohexylamino)ethanesulfonic acid (CHES), N-(1,1-Dimethyl-2-hydroxyethyl)-3-amino-2-hydroxypropanesulfonic acid (AMPSO) Piperazine-1,4-bis(2-hydroxypropanesulfonic acid) dihydrate, Piperazine-N,N′-bis(2-hydroxypropanesulfonic acid) (POPSO).

44. The kit of claim 42, wherein concentration of the reagent of formula (I) is from about 0.01 M to about 0.1 M; concentration of acetonitrile is from about 5%-50%, concentration of copper is from about 0.25 mM to about 0.5 mM.

45. The kit of claim 43, wherein concentration of tartrate is from about 5.7 mM to about 22.7 mM, concentration of sodium bicarbonate, potassium bicarbonate or sodium potassium bicarbonate is from about 0.01-0.2 M.

46. The kit of claim 42, further comprising one or more stop solutions packaged in a separate container, the stop solutions comprising acetic acid, citric acid, formic acid, hydrochloric acid, or sulphuric acid.

47. The kit of claim 42, further comprising a signal enhancer packaged in a separate container, the signal enhancer comprising a metal chelator selected from comprising Nitrilotriacetic acid (NTA), Ethylenediamine tetraacetic acid (EDTA), Iminodiacetic acid (IDA) or triscarboxymethyl ethylene diamine (TED).

48. The kit of claim 42, further comprising one or more stop solution and one or more signal enhancer packaged together in a separate container.

49. The kit of claim 42, wherein the pH of the composition, in use, is from about 11-12.2.
